(19) United States
(12) Patent Application Publication
(10) Pub. No.: US 2021/0114029 A1
(43) Pub. Date: Apr. 22, 2021

(54) DEVICES, SYSTEMS, AND METHODS FOR HIGH THROUGHPUT SINGLE CELL ANALYSIS

(71) Applicant: Duke University, Durham, NC (US)

(72) Inventors: Benjamin YELLEN, Durham, NC (US); Ying Li, Durham, NC (US); Jeff Motschman, Durham, NC (US)

(21) Appl. No.: 16/645,285

(22) PCT Filed: Oct. 17, 2018
(86) PCT No.: PCT/US2018/056221

§ 371 Date: Mar. 6, 2020

Related U.S. Application Data
(60) Provisional application No. 62/574,865, filed on Oct. 20, 2017.

(51) Int. Cl. B01L 3/00; C12M 3/06

(52) U.S. Cl. CPC B01L 3/502761; B01L 3/502746; C12M 23/16; B01L 2400/086; B01L 2300/0864; B01L 2400/0487; B01L 2300/087; B01L 2200/0668

(57) ABSTRACT

The present disclosure comprises devices, systems and methods for organizing cells into an array, phenotyping them via image-based analysis over short or long durations, and conducting massively parallel barcoded genomic analysis with DNA barcodes that are present next to each cell.

### CROSS-REFERENCE

This application claims the benefit of U.S. Provisional Application No. 62/574,865, filed on Oct. 20, 2017, which application is incorporated herein by reference.

### STATEMENT AS TO FEDERALLY SPONSORED RESEARCH

This invention was made with the support of the United States government under Federal Grant Nos. R2GM111584 and R01GM123542 awarded by the National Institutes of Health. The Federal Government has certain rights to this invention.

### BACKGROUND

Single cell analysis techniques may enable ground-breaking advances in a variety of basic research and clinical applications. For example, single cell analysis has the potential to enable rapid identification of rare, drug resistant cells in cases where conventional cell culture techniques require weeks or months of experimentation. However, no existing single cell analysis platform provides high capture efficiency in a cell trapping architecture that is compatible with the long-term cell culture, high-throughput microscopy, automated image processing, biochemical assay, and genomic analysis techniques that allow for large datasets to be efficiently analyzed. Thus, there is a need for improved methods of trapping and compartmentalizing single cells for subsequent phenotypic, biochemical, physiological, genetic, genomic, and/or proteomic analysis.

### SUMMARY

Disclosed herein are microfluidic devices comprising: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises a constriction in at least one dimension that is less than about one third of a smallest dimension of the object; and ii) a ratio of a fluidic resistance of a fluid flow path that bypasses a weir-trap to that for a fluid flow path passing through the weir-trap is at least 0.4.

In some embodiments, the ratio of fluidic resistance is at least 0.5. In some embodiments, the ratio of fluidic resistance is at least 0.75. In some embodiments, the ratio of fluidic resistance is at least 1.0. In some embodiments, the ratio of fluidic resistance is at least 1.25.

Also disclosed herein are microfluidic devices comprising: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises an entrance region, an interior region, and an exit region that collectively constitute an interior fluid flow path through the weir-trap that has a fluidic resistance, R_{T}; ii) each weir-trap in a majority of the weir-traps is in fluid communication with one long bypass fluid flow channel having a fluidic resistance, R_{A}, and with one or two short bypass fluid flow channels each having a fluidic resistance that is less than R_{A}, wherein each bypass fluid flow channel connects the exit region of the weir-trap to the entrance region of another weir-trap; and iii) a ratio R_{A}/R_{T} is at least 1.0.

Additionally, disclosed herein are microfluidic devices comprising: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises an entrance region, an interior region, and an exit region that collectively constitute an interior fluid flow path through the weir-trap that has a fluidic resistance, R_{T}; ii) each weir-trap in a majority of the weir-traps is in fluid communication with one long bypass fluid flow channel having a fluidic resistance, R_{A}, and with one or two short bypass fluid flow channels each having a fluidic resistance that is less than R_{A}, wherein each bypass fluid flow channel connects the exit region of the weir-trap to the entrance region of another weir-trap; and iii) fluid flows through an adjacent short bypass channel in a first direction if a weir-trap is unoccupied, and in a second direction if the weir-trap is occupied by an object.

In some embodiments, the ratio R_{A}/R_{T} is at least 1.1. In some embodiments, the ratio R_{A}/R_{T} is at least 1.2. In some embodiments, the ratio R_{A}/R_{T} is at least 1.3. In some embodiments, the ratio R_{A}/R_{T} is at least 1.4. In some embodiments, the ratio R_{A}/R_{T} is at least 1.45. In some embodiments, each weir-trap comprises at least one constriction that has a spatial dimension that is less than about one half of the smallest dimension of the object. In some embodiments, each weir-trap comprises at least one constriction that has a spatial dimension that is less than about one third of the smallest dimension of the suspended objects. In some embodiments, each weir-trap comprises at least one constriction that has a spatial dimension that ranges from about 1.5 μm to about 6 μm. In some embodiments, the ratio R_{A}/R_{T} is at least 1.2 and a capture probability for an individual weir-trap retaining a suspended object on first contact is at least 0.36. In some embodiments, the ratio R_{A}/R_{T} is at least 1.45 and a capture probability for an individual weir-trap retaining a suspended object on first contact is at least 0.60. In some embodiments, each weir-trap comprises a frit structure within the exit region, and wherein the frit structure comprises one or more constrictions that have a spatial dimension that is smaller than the smallest dimension of the suspended objects. In some embodiments, the plurality of weir-traps comprises at least 100 weir traps. In some embodiments, the plurality of weir-traps comprises at least 1,000 weir traps. In some embodiments, the plurality of weir-traps comprises at least 10,000 weir traps. In some embodiments, a pre-saturation trapping efficiency for trapping the suspended objects is at least 20%. In some embodiments, the plurality of weir-traps comprises at least 100,000 weir traps. In some embodiments, a pre-saturation trapping efficiency for trapping the suspended objects is at least 50%. In some embodiments, a pre-saturation trapping efficiency for trapping the suspended objects is at least 80%. In some embodiments, a pre-saturation trapping efficiency for trapping the suspended objects is at least 90%. In some embodiments, a pre-saturation trapping efficiency for trapping the suspended objects is at least 95%. In some embodiments, a pre-saturation trapping efficiency for trapping the suspended objects is at least 98%. In some embodiments, the microfluidic device further comprises: b) a removable lid. In some embodiments, an interior region of one or more weir-traps comprises a unique molecular identifier (or barcode) that may be bound to or hybridized to molecular components of a cell upon lysis of a cell within the interior region of a weir-trap.

Disclosed herein are methods for trapping objects suspended in a fluid, the methods comprising: a) providing a microfluidic device of any embodiments described herein; and b) flowing a fluid comprising the objects through the microfluidic device to trap objects in one or more of the plurality of weir-traps.

In some embodiments, each weir-trap comprises a frit structure within an exit region, and wherein the frit structure comprises one or more constrictions that have a spatial dimension that is smaller than the smallest dimension of the objects. In some embodiments, the flowing in (b) is performed at a first hydrodynamic pressure, thereby trapping an object in a constriction in an entrance region of one or more weir-traps. In some embodiments, the objects comprise deformable objects, and wherein the method further comprises subjecting the object(s) trapped in the constriction in the entrance region(s) of one or more weir-traps to a second hydrodynamic pressure that is higher than the first hydrodynamic pressure, thereby forcing the deformable object(s) through the constriction in the entrance region(s) and into an interior region of the one or more weir-traps. In some embodiments, the first hydrodynamic pressure ranges from about 1 to about 100 mbar. In some embodiments, the second hydrodynamic pressure ranges from about 100 mbar to about 1,000 mbar. In some embodiments, the ratio of the second hydrodynamic pressure to the first hydrodynamic pressure ranges from about 10× to about 20×. In some embodiments, the objects are cells or beads. In some embodiments, the flowing in (b) is repeated at least once, thereby allowing at least two objects to be confined within the interior region(s) of one or more weir-traps. In some embodiments, the flowing in (b) is repeated at least once using a fluid that comprises the same objects as that used in the first instance. In some embodiments, the flowing in (b) is repeated at least once using a fluid that comprises different objects than that used in the first instance. In some embodiments, the at least two objects confined within the interior region(s) of one or more weir-traps comprise at least two of the same cells, at least two different cells, at least two of the same beads, at least two different beads, or at least one cell and one bead. In some embodiments, the method further comprises sealing the plurality of weir-traps by flowing an immiscible fluid through the microfluidic device. In some embodiments, the immiscible fluid is oil or air. In some embodiments, the objects are cells, and the cells are cultured within the interior region(s) of the one or more weir-traps for a period of one or more days. In some embodiments, the cells are cultured within the interior region(s) of the one or more weir-traps for a period of one or more weeks. In some embodiments, the cells are cultured within the interior region(s) of the one or more weir-traps for a period of one or more months. In some embodiments, the objects are cells, and wherein the method further comprises the use of an imaging technique to phenotype cells within the interior region(s) of the one or more weir-traps. In some embodiments, the imaging technique is selected from the group consisting of bright-field imaging, fluorescence imaging, two-photon fluorescence imaging, or any combination thereof. In some embodiments, the interior regions of the plurality of weir-traps each comprise unique molecular identifiers that may be bound or hybridized to molecular components of a cell upon lysis of a cell within the interior region of a weir-trap. In some embodiments, the molecular components comprise proteins, peptides, DNA molecules, RNA molecules, mRNA molecules, or any combination thereof. In some embodiments, the unique molecular identifiers (or barcodes) are used to perform DNA sequencing, gene expression analysis, or chromatin analysis. In some embodiments, an externally-applied electric field is used to facilitate hybridization of nucleic acid molecular components to the unique molecular identifiers. In some embodiments, the microfluidic device further comprises a removable lid. In some embodiments, the deformable objects are cells, and following the trapping of cell(s) in the interior region(s) of one or more weir-traps, a biocompatible hydrogel is infused into the microfluidic device and allowed to polymerize. In some embodiments, following the polymerization of the hydrogel, the lid of the microfluidic device is removed to allow access to the trapped cells. In some embodiments, the biocompatible hydrogel is used to confine the genomic material of a trapped cell upon lysis of the cell.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a microfluidic device comprising a ladder-like network of trapping features (constrictions) and interconnecting bypass fluid channels.

FIGS. 2A and 2B illustrate two different flow regimes in microfluidic devices of similar design comprising a ladder-like network of trapping features and interconnecting fluid bypass channels. In this non-limiting example, the trapping features comprise frits in their exit regions.

FIG. 2A illustrates the flow through the device when the internal flow path through a trapping feature has a higher hydrodynamic flow resistance than that for a serpentine bypass fluid channel.

FIG. 2B illustrates the flow through the device when the internal flow path through a trapping feature has a lower hydrodynamic flow resistance than that for a serpentine bypass fluid channel.

FIG. 3 illustrates the equivalent resistance circuit for the ladder-like networks of trapping features and interconnecting fluid channels shown in FIG. 1 and FIGS. 2A and 2B.

FIGS. 4A and 4B illustrate two different flow regimes in microfluidic devices of similar design comprising a mesh-like network of trapping features and interconnecting fluid channels.

FIG. 4A illustrates the flow through the device when the internal flow path through a trapping feature has a higher hydrodynamic flow resistance than that for a serpentine bypass fluid channel.

FIG. 4B illustrates the flow through the device when the internal flow path through a trapping feature has a lower hydrodynamic flow resistance than that for a serpentine bypass fluid channel.

FIG. 5 illustrates the equivalent resistance circuit for the mesh-like network of trapping features and interconnecting fluid channels shown in FIGS. 4A and 4B.

FIG. 6 illustrates a mesh network trapping geometry that has a trapping ratio that is approximately calculated as: R_{A}/R_{T}=0.42

FIG. 7 illustrates a mesh network trapping geometry that has a trapping ratio that is approximately calculated as: R_{A}/R_{T}=1.2.

FIG. 8 illustrates a ladder network trapping geometry that has a trapping ratio that is approximately calculated as: R_{A}/R_{T}=1.2.

FIG. 9 illustrates a mesh network trapping geometry that has a trapping ratio that is approximately calculated as: R_{A}/R_{T}=1.45.

FIG. 10 illustrates a ladder network trapping geometry that has a trapping ratio that is approximately calculated as: R_{A}/R_{T}=1.45.

FIG. 11 illustrates a mesh network trapping geometry where the weir-traps comprise an interior flow path with a small volume (i.e., the traps have no significant “interior region”).

FIG. 12 illustrates one non-limiting example of a ladder network trapping geometry that has an interior flow path that does not have frits at the back side.

FIG. 13 illustrates one non-limiting example of a mesh network trapping geometry that has an interior flow path that does not have frits at the back side.

FIG. 14 provides a schematic illustration of an artificial neural network.

FIG. 15 provides a schematic illustration of the functionality of a node within a layer of an artificial neural network.

FIGS. 16A-16D show plots of capture percentage vs. row number for four different microfluidic devices comprising different ratios of the flow resistance through internal flow paths through trapping features and serpentine bypass fluid channels. FIG. 16A: plot for a microfluidic device in which the ratio of hydrodynamic flow resistance through a serpentine bypass channel to that for the flow path through a trapping feature (R_{A}/R_{T})=0.25. FIG. 16B: plot for a microfluidic device for which R_{A}/R_{T}=0.42. FIG. 16C: plot for a microfluidic device for which R_{A}/R_{T}=1.20.

FIG. 16D: plot for a microfluidic device for which R_{A}/R_{T}=1.45.

FIGS. 17A-17D show heat maps of the distribution of occupied traps for the four microfluidic devices that exhibit the capture percentage curves shown in FIGS. 16A-16D. FIG. 17A: heat map for a microfluidic device for which R_{A}/R_{T}=0.25. FIG. 17B: heat map for a microfluidic device for which R_{A}/R_{T}=0.42. FIG. 17C: heat map for a microfluidic device for which R_{A}/R_{T}=1.20. FIG. 17D: heat map for a microfluidic device for which R_{A}/R_{T}=1.45.

FIG. 18 shows a series of time lapse images of a single cell colony growing inside a microfluidic chamber. The centers of the cells are identified using a machine learning-based image processing algorithm, and are depicted as small dots.

FIG. 19 provides a non-limiting example of growth curves obtained using a machine learning-based analysis of images of cells grown within a microfluidic device of the present disclosure.

FIG. 20 show plots of growth rate data for K562 cells grown in a microfluidic device of the present disclosure, including data for a control and for cells grown in the presence of 0.1 uM, 0.3 uM, and 0.5 uM Imatinib.

FIG. 21 shows a series of time lapse images of four cell colonies growing inside adjacent microfluidic chambers.

FIGS. 22A and 22B show images of MOLM 13 cells grown in the presence of Quizartinib (FIG. 22A) or a control medium (FIG. 22B). A single clone is observed to grow out in the presence of the drug.

FIGS. 23A and 23B illustrate the use of image segmentation-based machine learning algorithms to identify individual cells as well as identifiers and markers on the microfluidic chip.

FIG. 23A: bright-field image. FIG. 23B: a computer-generated color image is overlaid on the bright-field image, and shows the identification of markers on the chip, different instances of cells that have been classified using a machine learning-based analysis, the boundaries of the individual cells, and quality scores of the degree of confidence in the prediction of whether the object detected is a cell.

FIG. 24 shows and image of an array of single cells trapped within microfluidic chambers, after which air is blown through the fluid channels to seal the chambers.

FIG. 25 shows an overlay of fluorescent and bright-field images that shows the hybridization of fluorescently-labeled target probes to oligonucleotide capture probes that are patterned inside the microfluidic chips.

FIGS. 26A-26C illustrate a process for forming single cell arrays. Single cell arrays are formed by flowing cells into an array along with a curable hydrogel (FIG. 26A), after which the lid can be peeled away (FIG. 26B) to provide access to the sample (FIG. 26C).

FIGS. 27A and 27B provide a non-limiting example of a microfluidic device comprising multiple trapping features for the capture of single cells or other objects suspended in a fluid. FIG. 27A: photograph of a microfluidic device comprising a 100×100 array of trapping features and microfluidic chambers. FIG. 27B: micrograph of the trapping features and fluid chambers within a microfluidic device of the present disclosure.

FIGS. 28A-28D provide examples of the flow profile through a trap for a low efficiency trapping device that was used in proof-of-principle work, as well as data for single cell trapping efficiency. FIG. 28A: calculated fluid flow velocity through a single trap of the device. FIG. 28B: micrograph showing a single trap of the device. FIG. 28C: heatmap showing the single cell trapping efficiency for the 10,000 compartments within the device. FIG. 28D: pie chart showing the distribution of microfluidic chambers within which 0, 1, 2, or 3 or more cells were trapped.

FIG. 29 shows a stitched fluorescent image of a cell array (cells are labeled with FITC cell tracker dye). Inset: enlarged overlay of fluorescent and bright-field images showing individual cells trapped within the device.

FIGS. 30A-30C show non-limiting examples of images that demonstrate the ability to print chemicals to specific cells in the array, which is made possible by the open architecture of the microfluidic device. FIG. 30A: two side by side patterns printed within a single cell array using a fluorescent label. FIG. 30B: pattern printed to specific cells within a cell array using a fluorescent label. FIG. 30C: pattern printed to specific cells within a cell array using a fluorescent label.

### DETAILED DESCRIPTION

The present disclosure provides novel microfluidic device designs based on mesh-like networks of cell trapping features and interconnecting fluid channels that enable highly efficient trapping of single cells or other objects suspended in a fluid, and that are compatible with on-chip cell compartmentalization and culturing techniques, high throughput microscopy and automated image processing techniques, and biochemical assay or genomic analysis techniques.

In one aspect, the disclosed microfluidic devices enable highly efficient trapping of single cells or other objects by employing designs that exploit a previously unrecognized trait of ladder and mesh fluidic networks. By tuning the relative fluidic resistances of flow paths in a hydrodynamic fluidic circuit comprising a plurality of trapping features and at least two different types of interconnecting bypass channels, the direction of flow of the fluid within the nearest bypass channels is towards (rather than away from) the cell traps such that every cell or object is forced into the first trap that it encounters.

In another aspect, the disclosed microfluidic devices enable compartmentalization of single cells and short-term or long-term on-chip cell culturing by employing weir-trap designs that comprise an entrance region, an optional interior region, and an exit region that collectively constitute an interior fluid flow path through the weir-trap. In some aspects, the interior region has a dimension and/or volume that is larger than the cells or objects to be trapped, and thus may be used for compartmentalization and/or culturing of single cells. Methods for trapping cells or objects within the entrance regions of a plurality of traps (e.g., using a relatively low hydrodynamic pressure drop across the device to drive fluid flow), and subsequently forcing the trapped cells or objects into the interior regions of the plurality of traps (e.g., using a pulse of relatively high hydrodynamic pressure) are also described.

In some aspects of the present disclosure, single cells or objects that have been trapped within the entrance regions or the interior regions of weir-traps may be further isolated or compartmentalized by flowing an immiscible fluid (e.g., oil or air) through the device following the trapping step. In some aspects, such isolation steps may be used to further facilitate subsequent biochemical, physiological, genetic, genomic, and/or proteomic analysis of trapped cells.

In some aspects of the present disclosure, the disclosed microfluidic single cell trapping devices may comprise a removable lid, and single cells or objects that have been trapped within the entrance regions or the interior regions of weir-traps may be further isolated or compartmentalized by flowing the soluble components required for formation of a semi-porous hydrogel into the device and then triggering a polymerization step. Removal of the lid then enables direct access to individual cells (or other objects) within the array of traps to facilitate subsequent biochemical, physiological, genetic, genomic, and/or proteomic analysis. In some aspects, removal of the lid to enable direct access to individual cells (or other objects) within the array of traps may be used to facilitate removal of selected cells (or other objects) from the array.

In some aspects of the present disclosure, machine learning-based image analysis may be used to identify and classify individual cells that have been trapped within an array of weir-traps based on phenotypic traits.

In some aspects of the present disclosure, the interior regions of the weir-traps in the microfluidic single cell trapping devices may comprise a set of pre-selected capture or detection reagents (e.g., antibodies directed to specific cell surface antigens) or barcoding reagents (e.g., oligonucleotide barcodes) that have been tethered, immobilized, synthesized, or printed within the weir-traps. For example, in some aspects the disclosed microfluidic devices may enable massively parallel barcoding for genomic analysis of single cells by printing DNA barcodes next to each cell, as will be discussed in more detail below.

The microfluidic devices, and associated methods and systems, provided herein thus allow for parallel single cell analysis at each step, including but not limited to: (1) methods for organizing an array of cells (and/or other objects) at high density, and capturing a majority of the cells transferred into a device; (2) methods for compartmentalizing single cells in impermeable or semi-permeable containers, or trapping them inside a semi-porous hydrogel; (3) methods for phenotyping cells via high resolution image-based analysis over short or long periods of time; and (4) methods for performing subsequent biochemical, physiological, genetic, genomic, and/or proteomic analysis. The disclosed methods, devices, and systems are enabling for a variety of basic research and clinical applications. For example, they may potentially be used to implement new approaches to validating drug safety and efficacy, or new methods for selecting better patent therapies. The disclosed methods, devices, and systems can be used for conducting highly parallel experiments which are necessary to identify and analyze the heterogeneity in cellular behavior, and in particular the identification of rare outliers that have clinical relevance. For example, the rare fraction of cells that are resistant to a drug are a strong indicator of the tendency of that drug treatment to enable the outgrowth of drug resistant clones, leading to tumor recurrence. Likewise, the disclosed methods, devices, and systems can be used to study heterogeneity in stem cell differentiation during exposure to different biochemical signaling molecules and other chemical agents. The disclosed methods, devices, and systems can also be used to study the interactions between different types of cells, such as immune cells interacting with cancer cells in the presence of checkpoint inhibitors, and other antibody therapies. The disclosed methods, devices, and systems can also be used to quickly identify cells that are particularly adept at producing desired proteins, enzymes, or other biological products. The disclosed methods, devices, and systems can also be used to establish multi-parameter datasets that includes both the functional measurements described above, and is linked to genomic measurements from those same cells or single cell derived colonies. The types of genomic measurements that can be conducted on these cells include mRNA expression analysis, antibody receptor analysis, DNA mutation analysis, splice variant analysis, epigenetic assays based on chromatin restriction, methylation states, as well as higher order chromosomal arrangements.

Various aspects of the methods, devices, and systems described herein may be applied to any of the particular applications set forth below or for any other types of single cell analysis applications. It shall be understood that different aspects of the disclosure can be appreciated individually, collectively, or in combination with each other.

### Definitions

Unless otherwise defined, all technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term ‘about’ a number refers to that number plus or minus 10% of that number. The term ‘about’ when used in the context of a range refers to that range minus 10% of its lowest value and plus 10% of its greatest value.

As used herein, the terms “trap”, “trapping feature”, “cell trap”, and “weir-trap” are used interchangeably, and may refer to a feature comprising a constriction in one or two dimensions within a fluid channel that is designed to retain or trap cells or other objects suspended in a fluid. In some instances, a trap may comprise an entrance region, optionally, an interior region, and an exit region, at least one of which comprises a constriction. In some instances an interior region of the trap may be significantly larger in at least one or two dimensions than the entrance region and/or exit region, and may be configured to compartmentalize individual cells that have been trapped.

As used herein, the term “object” generally refers to a cell or fragment thereof (e.g., a cellular organelle such as a cell nucleus, mitochondrion, or exosome), an organism (e.g., a bacterium), a bead, a particle, a droplet (e.g., a liquid droplet), or in plural form, may refer to any combination thereof.

As used herein, the term “cell” generally refers to any of a variety of cells known to those of skill in the art. In some aspects, the term “cell” may refer to any adherent and non-adherent eukaryotic cell, mammalian cell, a primary or immortalized human cell or cell line, a primary or immortalized rodent cell or cell line, a cancer cell, a normal or diseased human cell derived from any of a variety of different organs or tissue types (e.g., a white blood cell, red blood cell, platelet, epithelial cell, endothelial cell, neuron, glial cell, astrocyte, fibroblast, skeletal muscle cell, smooth muscle cell, gamete, or cell from the heart, lungs, brain, liver, kidney, spleen, pancreas, thymus, bladder, stomach, colon, small intestine), a distinct cell subset such as an immune cell, a CD8⁺ T cell, CD4⁺ T cell, CD44^{high}/CD24^{low} cancer stem cell, Lgr5/6⁺ stem cell, undifferentiated human stem cell, a human stem cell that has been induced to differentiate, a rare cell (e.g., a circulating tumor cell (CTC), a circulating epithelial cell, a circulating endothelial cell, a circulating endometrial cell, a bone marrow cell, a progenitor cell, a foam cell, a mesenchymal cell, or a trophoblast), an animal cell (e.g., mouse, rat, pig, dog, cow, or horse), a plant cell, a yeast cell, a fungal cell, a bacterial cell, an algae cell, an adherent or non-adherent prokaryotic cell, or in plural form, any combination thereof. In some aspects, the term “cell” may refer to an immune cell, e.g., a T cell, a cytotoxic (killer) T cell, a helper T cell, an alpha beta T cell, a gamma delta T cell, a T cell progenitor, a B cell, a B-cell progenitor, a lymphoid stem cell, a myeloid progenitor cell, a lymphocyte, a granulocyte, a Natural Killer cell, a plasma cell, a memory cell, a neutrophil, an eosinophil, a basophil, a mast cell, a monocyte, a dendritic cell, and/or a macrophage, or in plural form, to any combination thereof.

As used herein, the term “bead” generally refers to any type of solid, porous, or hollow spherical, non-spherical, or irregularly-shaped object composed of glass, plastic, ceramic, metal, a polymeric material, or any combination thereof. In some aspects, the term “bead” may refer to a silica bead, a silica gel bead, a controlled pore glass bead, a magnetic bead (e.g., a Dynabead), a Wang resin bead, a Merrifield resin bead, an agarose bead, a Sephadex bead, a Sepharose bead, a cellulose bead, a polystyrene bead, etc., or in plural form, may refer to any combination thereof. In some aspects, a bead may comprise tethered or immobilized capture, detection, or barcoding reagents, e.g., antibodies, cytokine-specific antibodies, chemokine-specific antibodies, growth factor-specific antibodies, enzymes, enzyme substrates, avidin or streptavidin, protein A, protein G, other proteins, small molecules, glycoproteins, drug molecules, polysaccharides, fluorophores, oligonucleotides, oligonucleotide aptamers, oligonucleotide barcodes, or any combination thereof. In some instances, a bead may be a cytokine-sensing bead such as multiplexed Luminex xMAP® immuno-assay beads sold by Thermo Fischer (Waltham, Mass.), which can be used to detect from 3 to 30 different cytokines and growth factors. In some aspects, the diameter or average diameter of a bead may be at least 0.5 μm, at least 1 μm, at least 5 μm, at least 10 μm, at least 15 μm, at least 20 μm, at least 25 μm, at least 30 μm, at least 35 μm, at least 40 μm, at least 45 μm, or at least 50 μm.

Microfluidic device designs for efficient trapping of single cells: As noted above, in one aspect the present disclosure provides microfluidic devices that enable highly efficient trapping of single cells or other objects by employing designs that exploit a previously unrecognized trait of mesh fluidic networks. Tuning the relative fluidic resistances of flow paths in a hydrodynamic fluidic circuit comprising a plurality of trapping features and at least two different types of interconnecting bypass channels ensures that all fluid flow streamlines go through the trap, thereby ensuring that every cell is forced into the first trap that it encounters. This phenomenon is achieved by adjusting the hydrodynamic resistance through the trap, R_{T} (i.e., the fluidic resistance of the entire trap geometry spanning the distance from the entry point to the exit point of a single trap) relative to the fluidic resistance through one or more short bypass channel sections, R_{B}, and a communal long bypass channel section, R_{A}, with the requirement that R_{T}<R_{A}. After a cell has been trapped, the local ratio of fluidic resistances changes in a manner such that the direction of fluid flow in the adjoining bypass channels reverses and flows away from the cell trap, thereby causing the next approaching cell to move towards the next available trap. In this manner, the traps within the array are populated sequentially in the order that cells are introduced, which in principle allows the disclosed devices to achieve near perfect efficiency in trapping single cells. The disclosed devices are thus ideally suited for handling small cell samples where high trapping efficiencies are critical.

The disclosed device designs are based on mesh-like networks of fluid channels. In some aspects, the devices comprise: a) a microfluidic network having at least one inlet and at least one outlet; b) a plurality of microfluidic constrictions (or “traps”), wherein a dimension of the constriction is smaller than a dimension of a suspended object contained within the fluid, and disposed so as to capture suspended objects flowing into the constriction; c) each microfluidic constriction comprising an entrance point or region and an exit point or region, and optionally, an interior region, d) the exit point of said microfluidic constriction is in direct fluidic connection with at least two additional microfluidic constrictions; e) the pressure at the exit point of said microfluidic constriction is higher than the pressure at the entrance point of either downstream microfluidic constriction when said microfluidic constriction has not yet captured a suspended object; and f) the pressure at the exit point of said microfluidic constriction is lower than the pressure at the entrance point of at least one of the downstream microfluidic constriction when said microfluidic constriction has captured a suspended object. In some aspects, the exit region of the constrictions or traps may comprise a frit, e.g., a series of columnar features having a spacing that is sufficiently small to prevent cells or other objects from leaving an interior region of the constriction or trap.

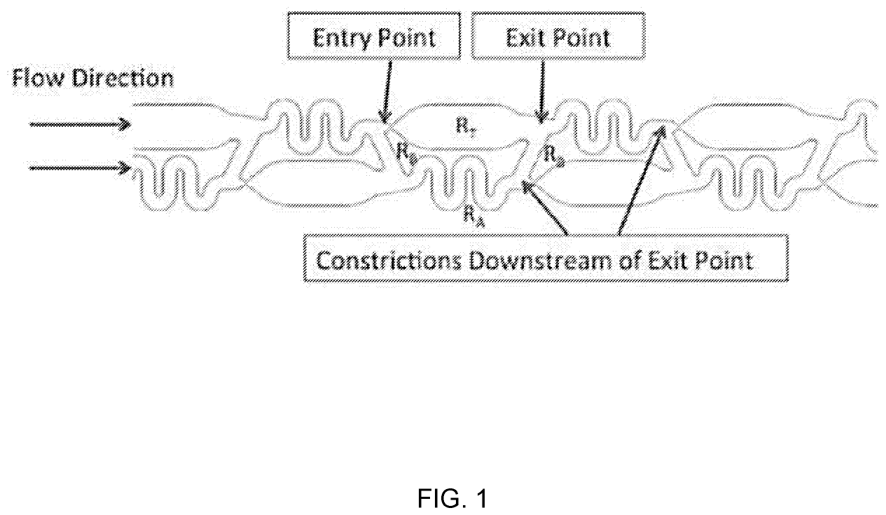

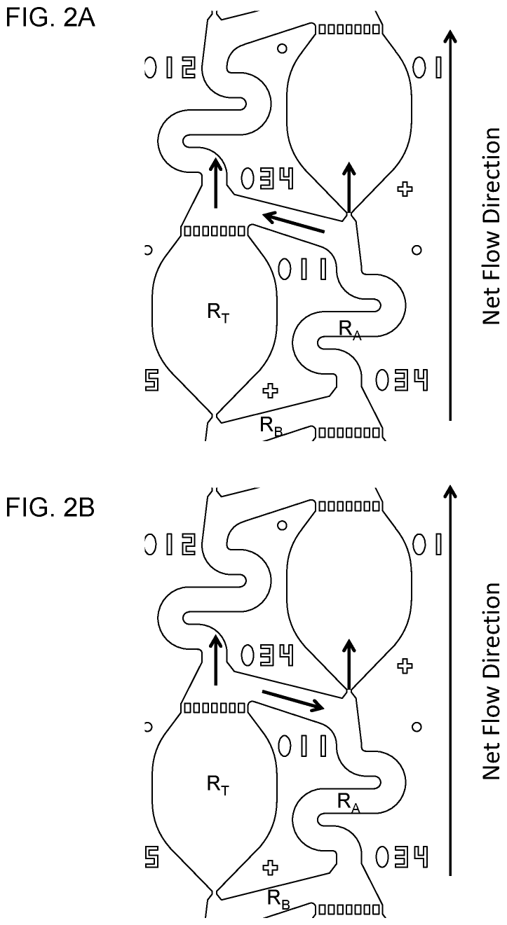

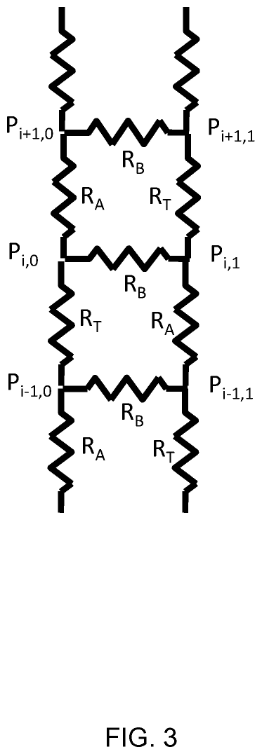

FIG. 1 illustrates a microfluidic device comprising an “infinite” ladder-like network of trapping features (each comprising a constricted entry point (or entrance region), an interior region, and an exit point) and an interconnecting set of bypass fluid channels. FIGS. 2A and 2B illustrate similar ladder-like fluidic networks where the trapping features each comprise a frit within the exit point (or exit region). The fluidic resistance of the flow path through the trap, R_{T}, comprises the fluidic resistance of the entire trap geometry spanning the distance from the entry point through an interior region of the trap to the exit point of the trap. Two types of bypass fluid channels are indicated in FIG. 1 and FIGS. 2A-B—a long, communal bypass channel comprising a fluidic resistance, R_{A}, (where, optionally, the bypass channel has a serpentine layout) and a shorter interconnecting bypass channel comprising a fluidic resistance, R_{B}. The long bypass channels comprising a fluidic resistance, R_{A}, are generally aligned with the direction of net flow through the device, while the short bypass channels comprising a fluidic resistance, R_{B}, are generally aligned perpendicularly to the direction of net flow through the device. In some instances, there may be more than one type of short bypass channel comprising fluidic resistances of R_{B1}, R_{B2}, . . . , where R_{B1}, R_{B2}, . . . , may be different from each other but will each be less than R_{A}. The equivalent resistor circuit for the fluidic devices illustrated in FIG. 1 and FIGS. 2A-B is shown in FIG. 3, and comprises a series of pressure nodes, P_{i,j}, linked by the fluidic resistances R_{T}, R_{A}, and R_{B}.

For the infinite ladder fluidic resistance network depicted in FIG. 3, the equations for current continuity are given by:

$$[ \begin{matrix}( {R}_{A}^{- 1} + {R}_{B}^{- 1} + {R}_{T}^{- 1} ) & - {R}_{B}^{- 1} & - ( {R}_{A}^{- 1} + {R}_{T}^{- 1} ) & 0 \\ - {R}_{B}^{- 1} & ( {R}_{A}^{- 1} + {R}_{B}^{- 1} + {R}_{T}^{- 1} ) & 0 & - ( {R}_{A}^{- 1} + {R}_{T}^{- 1} ) \\ - ( {R}_{A}^{- 1} + {R}_{T}^{- 1} ) & 0 & ( {R}_{A}^{- 1} + {R}_{B}^{- 1} + {R}_{T}^{- 1} ) & - {R}_{B}^{- 1} \\ 0 & - ( {R}_{A}^{- 1} + {R}_{T}^{- 1} ) & - {R}_{B}^{- 1} & ( {R}_{A}^{- 1} + {R}_{B}^{- 1} + {R}_{T}^{- 1} )\end{matrix} ] [ \begin{matrix}{P}_{i , 0} \\ {P}_{i , 1} \\ {P}_{i + 1 , 0} \\ {P}_{i + 1 , 1}\end{matrix} ] = Δ P [ \begin{matrix}{R}_{A}^{- 1} \\ {R}_{T}^{- 1} \\ - {R}_{A}^{- 1} \\ - {R}_{T}^{- 1}\end{matrix} ] \qquad ( 1 )$$

where ΔP is the pressure drop across one period of the ladder (from P_{i-1,1} to P_{i+1,1} or from P_{i-1,0} to P_{i+1,0}. The solution to this equation is given in terms of the pressure at the point, P_{i-1,0}:

$${P}_{i , 0} = {P}_{i , 0} \\ {P}_{i , 1} = {P}_{i , 0} - \frac{1}{2} \frac{{R}_{A}^{- 1} - {R}_{T}^{- 1}}{{R}_{A}^{- 1} + {R}_{B}^{- 1} + {R}_{T}^{- 1}} Δ P \\ {P}_{i + 1 , 0} = {P}_{i , 0} - \frac{1}{2} \frac{{R}_{B}^{- 1} + 2 {R}_{A}^{- 1}}{{R}_{A}^{- 1} + {R}_{B}^{- 1} + {R}_{T}^{- 1}} Δ P \\ {P}_{i + 1 , 1} = {P}_{i , 0} - \frac{1}{2} Δ P \qquad ( 2 )$$

This system of equations has two regimes of fluid flow. There is a regime where all the streamlines pass through the long channel section (comprising fluidic resistance R_{A}), and a fraction of the streamlines pass through the microfluidic constriction (comprising fluidic resistance R_{T}), with the remainder flowing through the short channel section (comprising fluidic resistance R_{B}), flowing in the direction away from the microfluidic constriction (FIG. 2A). This condition is achieved when the pressure at the microfluidic constriction entry point (P_{i,0}) is higher than the pressure at Po, which occurs when R_{A}<R_{T}.

In the other regime where R_{A}>R_{T}, the situation is reversed and all the streamlines pass through the microfluidic constriction (R_{T}), with the fluid flowing through the short channel sections (R_{B}) directed towards the microfluidic constriction (FIG. 2B). Thus, by adjusting the relative resistances of the trap and bypass channels, it is possible to ensure that cells will be moved into the constriction and trapped without some fraction or cells lost down the bypass, as is typically achieved with prior approaches.

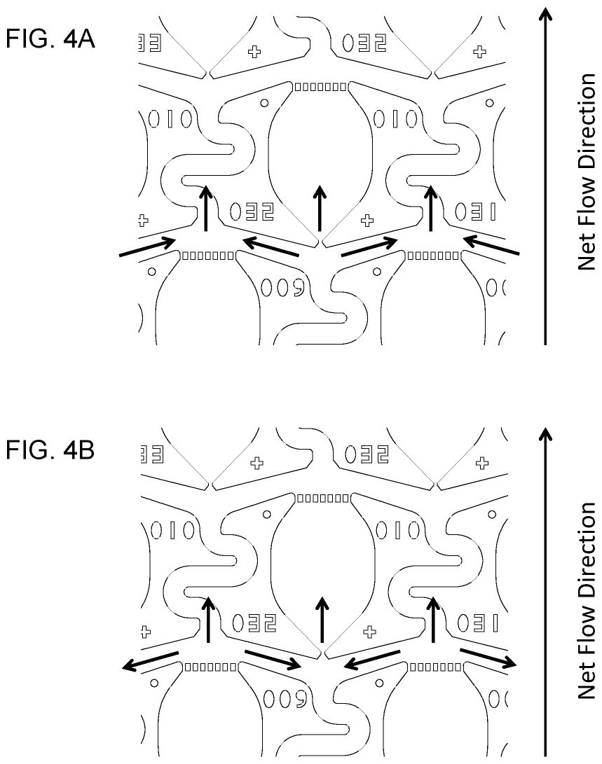

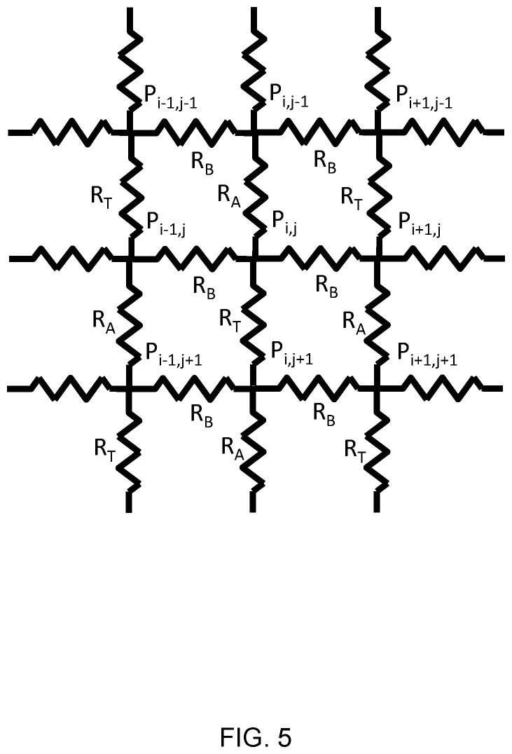

The analysis of a 2-D mesh network design (e.g., as illustrated in FIGS. 4A-B, which may be represented by the equivalent resistance circuit shown in FIG. 5) is similar to that discussed here for the infinite ladder network, and will be described in more detail in Example 1 below. The same condition, R_{A}>R_{T}, ensures that all the streamlines pass through the microfluidic constriction. This insight suggests that the first cell flowing through the array will be captured by the first available trap, and the next cell will populate the next available trap, and so on. Cells will never miss an unoccupied trap, and all traps will be populated in order.

In order to ensure that each trap captures only a single cell, it is also important to understand how the trap resistance changes once it becomes occupied by a cell, and what type of flow balance will be experienced by the next approaching cell. Ideally, the occupied trap would provide a flow profile in which the flow through the short bypass (R_{B}) is now larger than the flow through the trap. The flow ratio yields a condition:

R_{T}≥2R_{A}+2R_{B}

which could easily be achieved if the short channel section has very low resistance, while the presence of a trapped suspended object causes the trap resistance (R_{T}) to more than double. This insight implies that the depth of the channel should not be significantly larger than the cell diameter, such that a trapped cell occludes a significant cross-sectional area percentage of the microfluidic constriction and causes a maximal change in the trap resistance.

The disclosed ladder-like and mesh-like fluidic network designs constitute a novel and non-obvious improvement over prior microfluidic-based cell trapping devices. It is well established that a good cell trapping device will have high volumetric flow through the trap, and low volumetric flow around the trap, however, in contrast to previously published designs, we have recognized that the important design consideration is not the total pressure drop across the trap but rather the tuning of relative fluidic resistances for flow through a communal bypass channel and for flow through the trap in order to maintain the condition R_{A}>R_{T}.

FIGS. 2A, 2B, 4A, 4B, and 6-13 provide several different non-limiting examples of the ladder-like and mesh-like fluidic network designs of the present disclosure. As noted above, FIGS. 2A and 2B illustrate a ladder-like network of weir-traps (comprising frits in the exit region) and interconnecting bypass fluid channels. When the resistance of the internal flow path through the weir trap is higher than the resistance of the bypass channel (R_{T}>R_{A}; flow regime 1), the flow splits at the entrance to the weir trap (FIG. 2A). This geometry has lower trapping efficiency than that for flow regime 2, where the internal flow path through the weir trap has lower resistance than the flow path through the bypass channel (R_{A}>R_{T}), in which case all the fluid flows through the weir trap (FIG. 2B).

FIGS. 4A and 4B illustrate a mesh-like network of weir-traps and interconnecting bypass fluid channels. Again, when the resistance of the internal flow path through the weir trap is higher than the resistance of the bypass channel (R_{T}>R_{A}; flow regime 1), the flow splits at the entrance to the weir trap (FIG. 4A). When the internal flow path through the weir trap has lower resistance than the flow path through the bypass channel (R_{A}>R_{T}; flow regime 2), all of the fluid flows through the weir trap (FIG. 4B).

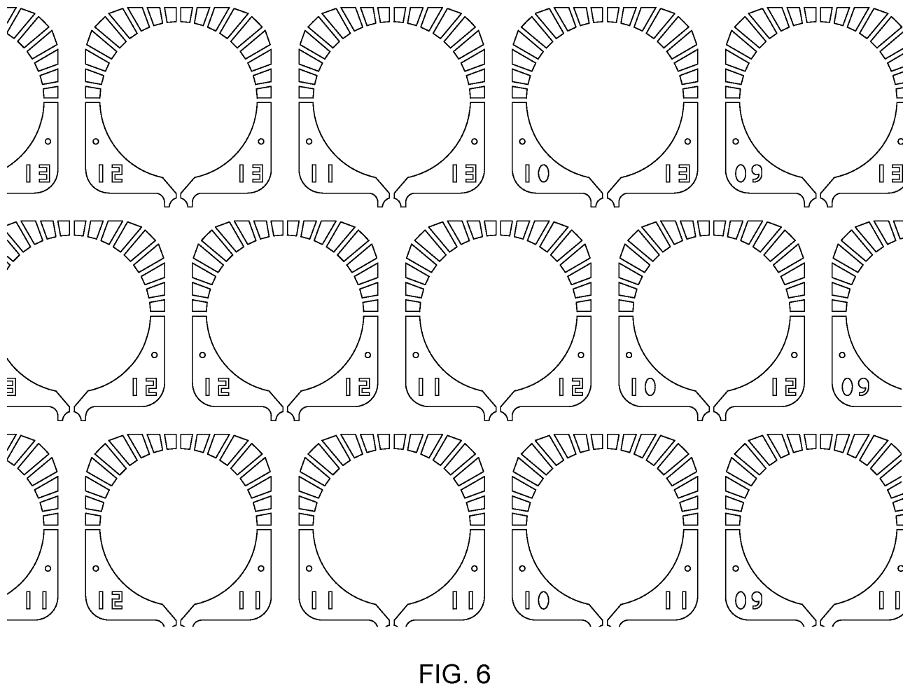

FIG. 6 illustrates a mesh network trapping geometry that has a trapping ratio that is approximately calculated as: R_{A}/R_{T}=0.42. In this example, the exit region of each weir-trap comprises a frit that forms the boundary of the interior region, and the interior region of the weir-trap is quite large in comparison to the entrance region comprising the constriction used to trap cells or objects suspended in a fluid.

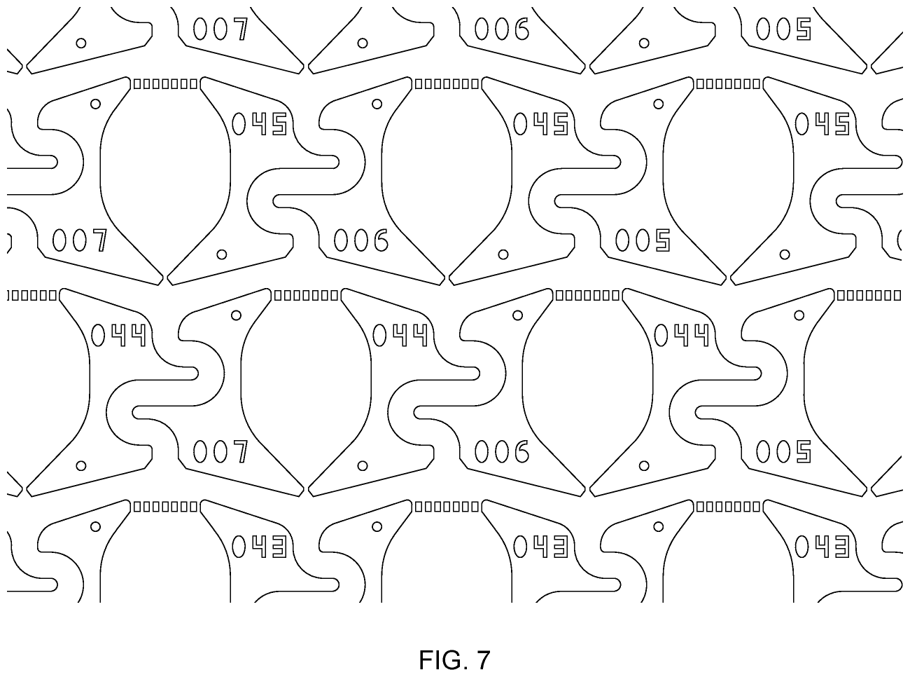

FIG. 7 illustrates a mesh network trapping geometry that has a trapping ratio that is approximately calculated as: R_{A}/R_{T}=1.2. The weir-traps in this example again comprise a frit within the exit region of the trap.

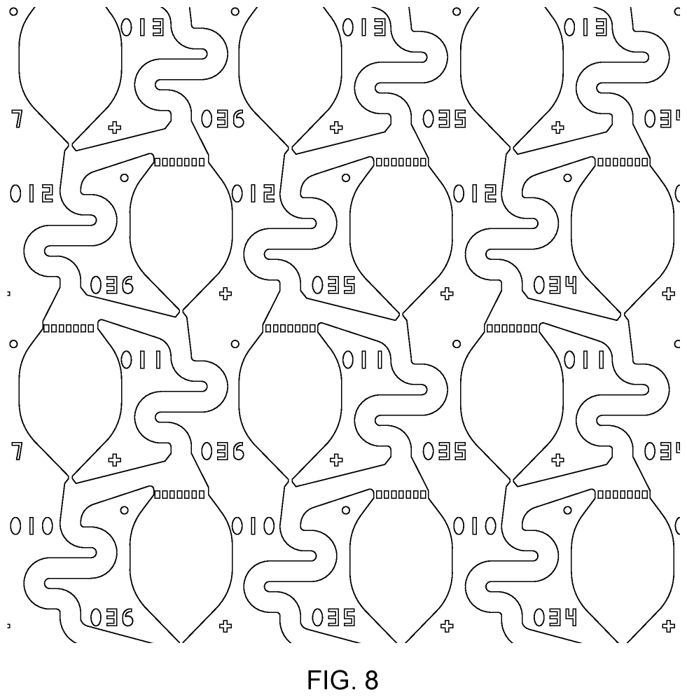

FIG. 8 illustrates a ladder network trapping geometry that has a trapping ratio that is approximately calculated as: R_{A}/R_{T}=1.2. The weir-traps in this example again comprise a frit within the exit region of the trap.

FIG. 9 illustrates a mesh network trapping geometry that has a trapping ratio that is approximately calculated as: R_{A}/R_{T}=1.45. The weir-traps in this example again comprise a frit within the exit region of the trap.

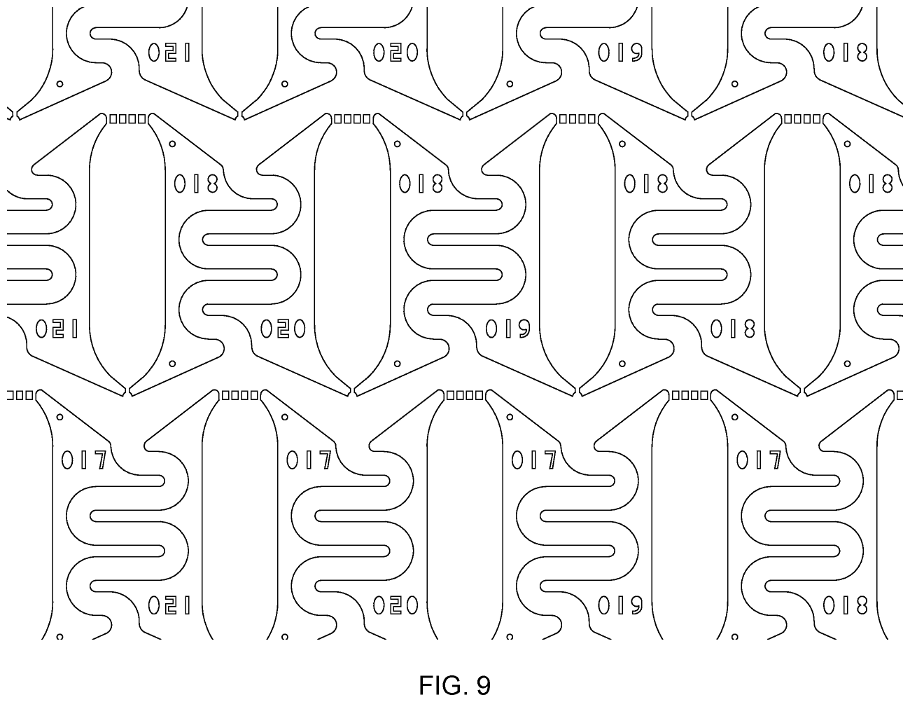

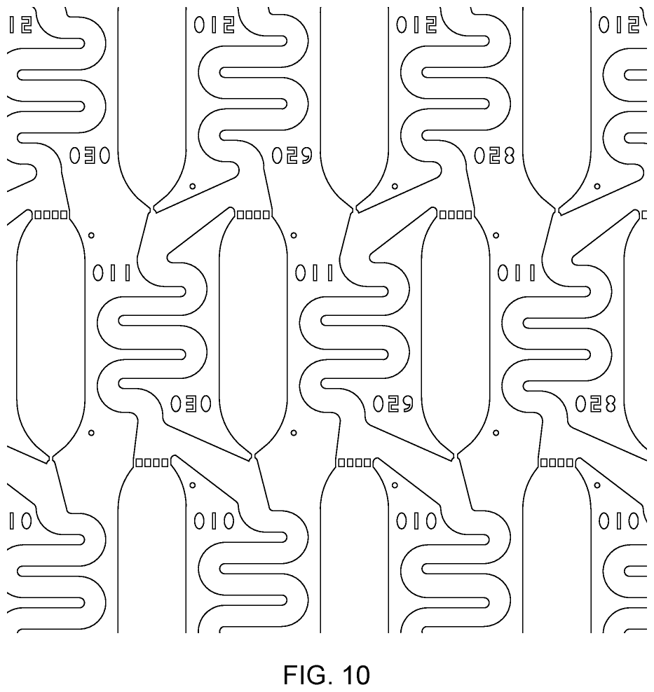

FIG. 10 illustrates a ladder network trapping geometry that has a trapping ratio that is approximately calculated as: R_{A}/R_{T}=1.45. The weir-traps in this example again comprise a frit within the exit region of the trap.

FIG. 11 illustrates a mesh network trapping geometry where the weir-traps comprise an interior flow path with a small volume (i.e., the traps have no significant “interior region”) and where the weir-traps lack a frit in the exit region of the trap.

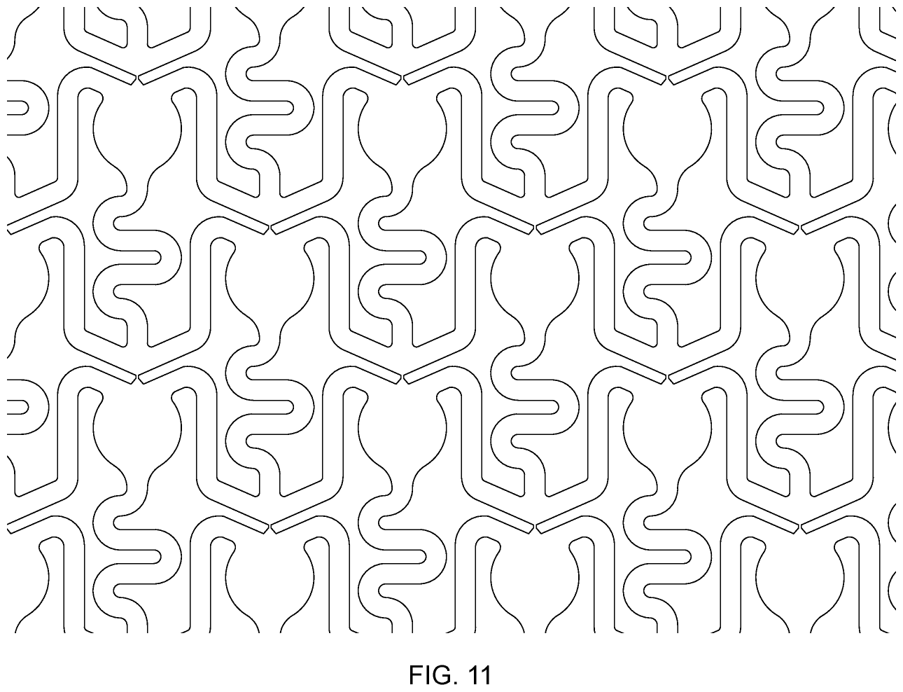

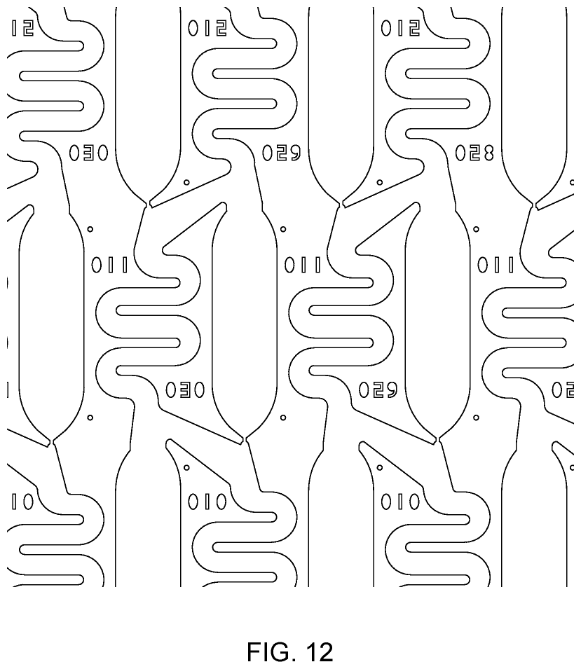

FIG. 12 illustrates one non-limiting example of a ladder network trapping geometry that has an interior flow path that does not have frits at the outlet or exit region.

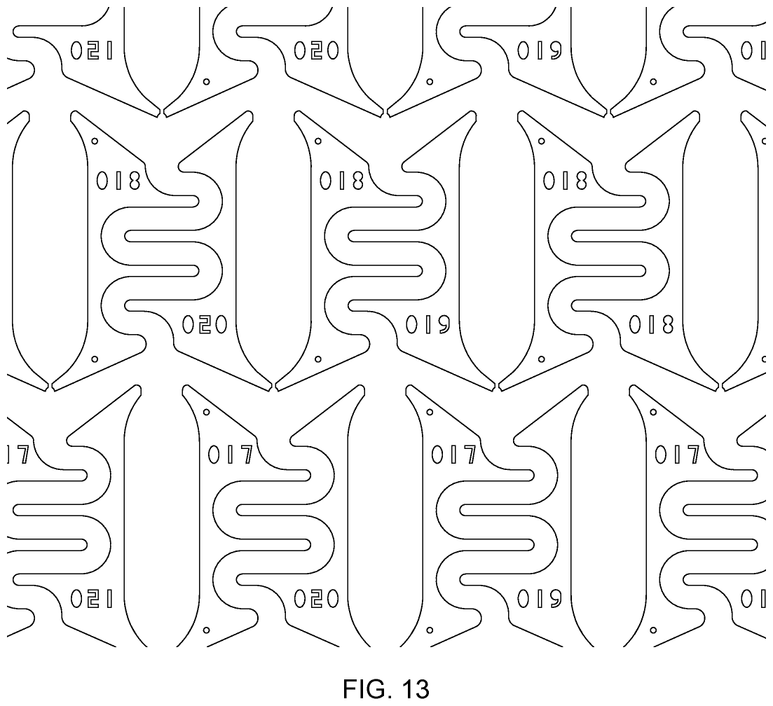

FIG. 13 illustrates one non-limiting example of a mesh network trapping geometry that has an interior flow path that does not have frits at the outlet or exit region.

In some instances, the disclosed microfluidic devices may comprise: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises an entrance region, an optional interior region, and an exit region that collectively constitute an interior fluid flow path through the weir-trap; ii) each weir-trap in a majority of the weir-traps (i.e., all of the weir-traps except for those nearest the at least one fluid inlet or at least one fluid outlet) is in fluid communication with either two or three exterior fluid flow paths (bypass fluid channels) that connect the exit region of a weir-trap to the entrance region of another weir-trap; and iii) a ratio of the fluidic resistance of one exterior fluid flow path (e.g., a longer, communal fluid bypass channel) to that of the interior fluid flow path through the trap (i.e., R_{A}/R_{T}) is at least 0.4. In some embodiments, the exit region of all or a portion of the weir-traps may comprise a frit to prevent cells or other objects from flowing out of the interior region (or chamber) of the trap. In some embodiments, the two or three exterior fluid flow paths (bypass fluid channels) may comprise one or two shorter fluid bypass channels comprising a fluidic resistance, R_{B}, which is less than R_{A}. In the case that there are two shorter fluid bypass channels, their fluidic resistance may be the same as each other, or different from each other, but will in either case be less than R_{A}.

In some embodiments, the ratio R_{A}/R_{T} may range from about 0.2 to about 2.0. In some embodiments, the ratio R_{A}/R_{T} may be at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. In some embodiments, the ratio R_{A}/R_{T} may be at most 2.0, at most 1.9, at most 1.8, at most 1.7, at most 1.6, at most 1.5, at most 1.4, at most 1.3, at most 1.2, at most 1.1, at most 1.0, at most 0.9, at most 0.8, at most 0.7, at most 0.6, at most 0.5, at most 0.4, at most 0.3, or at most 0.2. Any of the lower and upper values described in this paragraph may be combined to form a range included within the present disclosure, for example, the ratio R_{A}/R_{T} may range from about 0.4 to about 1.6. Those of skill in the art will recognize that the ratio R_{A}/R_{T} may have any value within this range, e.g., about 1.25.

The weir-traps of the disclosed microfluidic devices will generally comprise a constriction in at least one dimension, e.g., an entry point or entrance region comprising a constriction that is smaller than the smallest dimension of the cell or object to be trapped. In some embodiments, the constriction in at least one dimension may range in size from about 10% to about 90% of the smallest dimension of the cell or object to be trapped. In some embodiments, the constriction may be at least 10%, at least 20%, at least 30%, at least 40%, at least 50%, at least 60%, at least 70%, at least 80%, or at least 90% of the smallest dimension of the cell or object to be trapped. In some embodiments, the constriction may be at most 90%, at most 80%, at most 70%, at most 60%, at most 50%, at most 40%, at most 30%, at most 20%, or at most 10% of the smallest dimension of the cell or object to be trapped. Any of the lower and upper values described in this paragraph may be combined to form a range included within the present disclosure, for example the constriction may range in size from about 20% to about 70% of the smallest dimension of the cell or object to be trapped. Those of skill in the art will recognize that the constriction may have any value within this range, e.g., about 33% of the smallest dimension of the cell or object to be trapped.

The weir-traps of the disclosed microfluidic devices will generally comprise a constriction in at least one dimension, e.g., an entry point or entrance region comprising a constriction that is smaller than the smallest dimension of the cell or object to be trapped. In some embodiments, the constriction in at least one dimension may range in size from about 1 μm to about 100 μm. For example, in some embodiments, the constriction in at least one dimension may have a dimension of at least 1 μm, at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, or at least 100 μm. In some embodiments, the constriction in at least one dimension may have a dimension of at most 100 μm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm. Any of the lower and upper values described in this paragraph may be combined to form a range included within the present disclosure, for example the constriction in at least one dimension may range in size from about 3 μm to about 6 μm. Those of skill in the art will recognize that the constriction may have any dimension within this range, e.g., about 4.5 μm.

In some instances, the disclosed microfluidic devices may comprise: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises an entrance region, an interior region, and an exit region that collectively constitute an interior fluid flow path through the weir-trap; and ii) the volume of the interior region of the weir trap is greater than the volume of the entrance region or exit region.

In some instances, the disclosed microfluidic devices may comprise: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises an entrance region, an interior region, and an exit region that collectively constitute an interior fluid flow path through the weir-trap; and ii) the interior region has at least two dimensions that are greater than the largest dimension of the object.

The weir-trap designs of the disclosed microfluidic devices may comprise an entrance region (or entry point), optionally, an interior region (or chamber), and an exit region (or exit point). The interior region (or chamber), if present, may have any of a variety of cross-sectional shapes within the plane of the microfluidic device. For example, the interior region may have a largely circular shape, elliptical shape, square shape, rectangular shape, triangular shape, hexagonal shape, irregular shape, or any combination thereof. In some instances, the exit regions of all or a portion of the weir-traps may comprise a frit.

In some instances, the interior region may have negligibly small dimensions or volume relative to those of the entrance and/or exit regions of the trap. In some embodiments, the interior region (or chamber) may comprise a volume that ranges from 1× to about 1,000× that of the entrance region, exit region, or cell or object to be trapped. For example, in some embodiments, the interior region may comprise a volume that is at least 1×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1,000× of the entrance region, exit region, or cell or object to be trapped. In some embodiments, the interior region may comprise a volume that is at most 1,000×, at most 900×, at most 800×, at most 700×, at most 600×, at most 500×, at most 400×, at most 300×, at most 200×, at most 100×, at most 90×, at most 80×, at most 70×, at most 60×, at most 50×, at most 40×, at most 30×, at most 20×, at most 10×, or at most 1×that of the entrance region, exit region, or cell or object to be trapped. Any of the lower and upper values described in this paragraph may be combined to form a range included within the present disclosure, for example the interior region may comprise a volume that ranges in size from about 50× to about 200× that of the entrance region, exit region, or cell or object to be trapped. Those of skill in the art will recognize that the interior region may comprise a volume that has any value within this range, e.g., about 250× that of the entrance region, exit region, or cell or object to be trapped.

In some embodiments, the interior region (or chamber) may comprise at least one or at least two dimensions that range in size from about 1× to about 1,000× that of the largest dimension of the cell or object to be trapped. For example, in some embodiments, the interior region may comprise at least one or at least two dimensions that are at least 1×, at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, or at least 1,000× that of the largest dimension of the cell or object to be trapped. In some embodiments, the interior region may comprise at least one or at least two dimensions that are at most 1,000×, at most 900×, at most 800×, at most 700×, at most 600×, at most 500×, at most 400×, at most 300×, at most 200×, at most 100×, at most 90×, at most 80×, at most 70×, at most 60×, at most 50×, at most 40×, at most 30×, at most 20×, at most 10×, or at most 1× that of the largest dimension of the cell or object to be trapped. Any of the lower and upper values described in this paragraph may be combined to form a range included within the present disclosure, for example the interior region may comprise at least one or at least two dimensions that range in size from about 50× to about 200× that of the largest dimension of the cell or object to be trapped. Those of skill in the art will recognize that the interior region may comprise at least one or at least two dimensions that have any value within this range, e.g., about 125× that of the largest dimension of the cell or object to be trapped.

The capture probability for an individual weir-trap of the disclosed devices retaining a suspended cell or object on first contact (i.e., the first time that a cell or object encounters a weir-trap within the device) may range from about 0.05 to about 0.99. For example, in some embodiments, the capture probability may be at least 0.05, at least 0.1, at least 0.2, at least 0.3, at least 0.4, at least 0.5, at least 0.6, at least 0.7, at least 0.8, at least 0.9, at least 0.95, or at least 0.99. In some embodiments, the capture probability may be at most 0.99, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example the capture probability may range from about 0.2 to about 0.8. Those of skill in the art will recognize that the capture probability may have any value within this range, e.g., about 0.66.

The pre-saturation trapping efficiencies for trapping cells or other objects suspended in a fluid passing through the disclosed weir-trap array devices may range from about 10% to about 100%. For example, in some embodiments, the pre-saturation trapping efficiency of the disclosed devices may be at least 10%, at least 20%, at least 30%, at least 40%, at least 50%, at least 60%, at least 70%, at least 80%, at least 90%, at least 95%, at least 98%, or at least 99%. In some embodiments, the pre-saturation trapping efficiency may be at most 99%, at most 98%, at most 95%, at most 90%, at most 80%, at most 70%, at most 60%, at most 50%, at most 40%, at most 30%, at most 20%, or at most 10%. Any of the lower and upper values described in this paragraph may be combined to form a range included within the present disclosure, for example the pre-saturation trapping efficiency may range from about 40% to about 99%. Those of skill in the art will recognize that the pre-saturation trapping efficiency may have any value within this range, e.g., about 97%.

In some instances, the disclosed microfluidic devices may comprise: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) each weir-trap comprises a constriction in at least one dimension that is smaller than the smallest dimension of the object; and ii) a ratio of a fluidic resistance of a fluid flow path that bypasses a weir-trap to that for a fluid flow path passing through the weir-trap is at least 0.4. In some instances, as noted above, the constriction in at least one dimension may range in size from about 10% to about 90% of the smallest dimension of the cell or object to be trapped. For any of these instances in which the constriction in at least one dimension ranges in size from about 10% to about 90% of the smallest dimension of the cell or object to be trapped, the resistance of the fluid flow path that bypasses the weir-trap to that for the fluid flow path passing through the weir-trap (R_{A}/R_{T}) may range from about 0.4 to about 2.0. Non-limiting examples of combinations of constriction dimension (specified in terms of the percentage of the smallest dimension of the cell or object to be trapped) and resistance ratio (R_{A}/R_{T}) that are included in the present disclosure are (10%, 0.5), (10%, 0.6), (10%, 0.7), (10%, 0.8), (10%, 0.9), (10%, 1.0), (10%, 1.1), (10%, 1.2), (10%, 1.3), (10%, 1.4), (10%, 1.5), (10%, 1.6), (10%, 1.7), (10%, 1.8), (10%, 1.9), (10%, 2.0), (20%, 0.5), (20%, 0.6), (20%, 0.7), (20%, 0.8), (20%, 0.9), (20%, 1.0), (20%, 1.1), (20%, 1.2), (20%, 1.3), (20%, 1.4), (20%, 1.5), (20%, 1.6), (20%, 1.7), (20%, 1.8), (20%, 1.9), (20%, 2.0), (30%, 0.5), (30%, 0.6), (30%, 0.7), (30%, 0.8), (30%, 0.9), (30%, 1.0), (30%, 1.1), (30%, 1.2), (30%, 1.3), (30%, 1.4), (30%, 1.5), (30%, 1.6), (30%, 1.7), (30%, 1.8), (30%, 1.9), (30%, 2.0), (40%, 0.5), (40%, 0.6), (40%, 0.7), (40%, 0.8), (40%, 0.9), (40%, 1.0), (40%, 1.1), (40%, 1.2), (40%, 1.3), (40%, 1.4), (40%, 1.5), (40%, 1.6), (40%, 1.7), (40%, 1.8), (40%, 1.9), (40%, 2.0), (50%, 0.5), (50%, 0.6), (50%, 0.7), (50%, 0.8), (50%, 0.9), (50%, 1.0), (50%, 1.1), (50%, 1.2), (50%, 1.3), (50%, 1.4), (50%, 1.5), (50%, 1.6), (50%, 1.7), (50%, 1.8), (50%, 1.9), (50%, 2.0), (60%, 0.5), (60%, 0.6), (60%, 0.7), (60%, 0.8), (60%, 0.9), (60%, 1.0), (60%, 1.1), (60%, 1.2), (60%, 1.3), (60%, 1.4), (60%, 1.5), (60%, 1.6), (60%, 1.7), (60%, 1.8), (60%, 1.9), (60%, 2.0), (70%, 0.5), (70%, 0.6), (70%, 0.7), (70%, 0.8), (70%, 0.9), (70%, 1.0), (70%, 1.1), (70%, 1.2), (70%, 1.3), (70%, 1.4), (70%, 1.5), (70%, 1.6), (70%, 1.7), (70%, 1.8), (70%, 1.9), (70%, 2.0), (80%, 0.5), (80%, 0.6), (80%, 0.7), (80%, 0.8), (80%, 0.9), (80%, 1.0), (80%, 1.1), (80%, 1.2), (80%, 1.3), (80%, 1.4), (80%, 1.5), (80%, 1.6), (80%, 1.7), (80%, 1.8), (80%, 1.9), (80%, 2.0), (90%, 0.5), (90%, 0.6), (90%, 0.7), (90%, 0.8), (90%, 0.9), (90%, 1.0), (90%, 1.1), (90%, 1.2), (90%, 1.3), (90%, 1.4), (90%, 1.5), (90%, 1.6), (90%, 1.7), (90%, 1.8), (90%, 1.9), and (90%, 2.0).

In some instances, the disclosed microfluidic devices may comprise: a) a plurality of weir-traps disposed between, and in fluid communication with, at least one fluid inlet and at least one fluid outlet, wherein each weir-trap is configured to retain an object suspended in a fluid passing through the microfluidic device, and wherein: i) the capture probability for an individual weir-trap of retaining a suspended cell or object on first contact is at least 0.05; and ii) a ratio of a fluidic resistance of a fluid flow path that bypasses a weir-trap to that for a fluid flow path passing through the weir-trap is at least 0.4. In some instances, as noted above, the capture probability may range from about 0.05 to about 0.99. For some instances in which the capture probability ranges from about 0.05 to about 0.99, the resistance of the fluid flow path that bypasses the weir-trap to that for the fluid flow path passing through the weir-trap (R_{A}/R_{T}) may range from about 0.4 to about 2.0. In general, since the capture probability is a function of the resistance ratio, some combinations of capture probability and resistance ratio may not be achievable. Non-limiting examples of combinations of capture probability and resistance ratio (R_{A}/R_{T}) that may be included in the present disclosure are (0.05, 0.4), (0.05, 0.5), (0.05, 0.6), (0.05, 0.7), (0.05, 0.8), (0.05, 0.9), (0.05, 1.0), (0.05, 1.1), (0.05, 1.2), (0.05, 1.3), (0.05, 1.4), (0.05, 1.5), (0.05, 1.6), (0.05, 1.7), (0.05, 1.8), (0.05, 1.9), (0.05, 2.0), (0.1, 0.4), (0.1, 0.5), (0.1, 0.6), (0.1, 0.7), (0.1, 0.8), (0.1, 0.9), (0.1, 1.0), (0.1, 1.1), (0.1, 1.2), (0.1, 1.3), (0.1, 1.4), (0.1, 1.5), (0.1, 1.6), (0.1, 1.7), (0.1, 1.8), (0.1, 1.9), (0.1, 2.0), (0.2, 0.4), (0.2, 0.5), (0.2, 0.6), (0.2, 0.7), (0.2, 0.8), (0.2, 0.9), (0.2, 1.0), (0.2, 1.1), (0.2, 1.2), (0.2, 1.3), (0.2, 1.4), (0.2, 1.5), (0.2, 1.6), (0.2, 1.7), (0.2, 1.8), (0.2, 1.9), (0.2, 2.0), (0.3, 0.4), (0.3, 0.5), (0.3, 0.6), (0.3, 0.7), (0.3, 0.8), (0.3, 0.9), (0.3, 1.0), (0.3, 1.1), (0.3, 1.2), (0.3, 1.3), (0.3, 1.4), (0.3, 1.5), (0.3, 1.6), (0.3, 1.7), (0.3, 1.8), (0.3, 1.9), (0.3, 2.0), (0.4, 0.4), (0.4, 0.5), (0.4, 0.6), (0.4, 0.7), (0.4, 0.8), (0.4, 0.9), (0.4, 1.0), (0.4, 1.1), (0.4, 1.2), (0.4, 1.3), (0.4, 1.4), (0.4, 1.5), (0.4, 1.6), (0.4, 1.7), (0.4, 1.8), (0.4, 1.9), (0.4, 2.0), (0.5, 0.4), (0.5, 0.5), (0.5, 0.6), (0.5, 0.7), (0.5, 0.8), (0.5, 0.9), (0.5, 1.0), (0.5, 1.1), (0.5, 1.2), (0.5, 1.3), (0.5, 1.4), (0.5, 1.5), (0.5, 1.6), (0.5, 1.7), (0.5, 1.8), (0.5, 1.9), (0.5, 2.0), (0.6, 0.4), (0.6, 0.5), (0.6, 0.6), (0.6, 0.7), (0.6, 0.8), (0.6, 0.9), (0.6, 1.0), (0.6, 1.1), (0.6, 1.2), (0.6, 1.3), (0.6, 1.4), (0.6, 1.5), (0.6, 1.6), (0.6, 1.7), (0.6, 1.8), (0.6, 1.9), (0.6, 2.0), (0.7, 0.4), (0.7, 0.5), (0.7, 0.6), (0.7, 0.7), (0.7, 0.8), (0.7, 0.9), (0.7, 1.0), (0.7, 1.1), (0.7, 1.2), (0.7, 1.3), (0.7, 1.4), (0.7, 1.5), (0.7, 1.6), (0.7, 1.7), (0.7, 1.8), (0.7, 1.9), (0.7, 2.0), (0.8, 0.4), (0.8, 0.5), (0.8, 0.6), (0.8, 0.7), (0.8, 0.8), (0.8, 0.9), (0.8, 1.0), (0.8, 1.1), (0.8, 1.2), (0.8, 1.3), (0.8, 1.4), (0.8, 1.5), (0.8, 1.6), (0.8, 1.7), (0.8, 1.8), (0.8, 1.9), (0.8, 2.0), (0.9, 0.4), (0.9, 0.5), (0.9, 0.6), (0.9, 0.7), (0.9, 0.8), (0.9, 0.9), (0.9, 1.0), (0.9, 1.1), (0.9, 1.2), (0.9, 1.3), (0.9, 1.4), (0.9, 1.5), (0.9, 1.6), (0.9, 1.7), (0.9, 1.8), (0.9, 1.9), (0.9, 2.0), (0.95, 0.4), (0.95, 0.5), (0.95, 0.6), (0.95, 0.7), (0.95, 0.8), (0.95, 0.9), (0.95, 1.0), (0.95, 1.1), (0.95, 1.2), (0.95, 1.3), (0.95, 1.4), (0.95, 1.5), (0.95, 1.6), (0.95, 1.7), (0.95, 1.8), (0.95, 1.9), (0.95, 2.0), (0.99, 0.4), (0.99, 0.5), (0.99, 0.6), (0.99, 0.7), (0.99, 0.8), (0.99, 0.9), (0.99, 1.0), (0.99, 1.1), (0.99, 1.2), (0.99, 1.3), (0.99, 1.4), (0.99, 1.5), (0.99, 1.6), (0.99, 1.7), (0.99, 1.8), (0.99, 1.9), and (0.99, 2.0).

Microfluidic device fabrication: In some embodiments, the microfluidic devices disclosed herein may comprise at least two separately fabricated parts (e.g., (i) a substrate that incorporates etched, embossed, or ablated fluid channels, and (ii) a cover or lid) that are subsequently either mechanically clamped together, temporarily adhered together, or permanently bonded together. In some embodiments, the microfluidic devices disclosed herein may comprise three or more separately fabricated parts (e.g., (i) a substrate, (ii) a fluid channel layer, and (iii) a cover or lid) that are subsequently either mechanically clamped together, temporarily adhered together, or permanently bonded together. In some embodiments, the microfluidic devices disclosed herein may comprise a removable cover or lid. Examples of suitable fabrication techniques include, but are not limited to, conventional machining, CNC machining, injection molding, 3D printing, alignment and lamination of one or more layers of laser- or die-cut polymer film, or any of a number of microfabrication techniques such as photolithography and wet chemical etching, dry etching, deep reactive ion etching (DRIE), or laser micromachining. In some embodiments, all or a portion of the microfluidic devices may be 3D printed from an elastomeric material.

The microfluidic devices disclosed herein may be fabricated using any of a variety of materials known to those of skill in the art. In general, the choice of material used will depend on the choice of fabrication technique, and vice versa. Examples of suitable materials include, but are not limited to, silicon, fused-silica, glass, any of a variety of polymers, e.g. polydimethylsiloxane (PDMS; elastomer), polymethylmethacrylate (PMMA), polycarbonate (PC), polystyrene (PS), polypropylene (PP), polyethylene (PE), high density polyethylene (HDPE), polyimide, cyclic olefin polymers (COP), cyclic olefin copolymers (COC), polyethylene terephthalate (PET), epoxy resins, a non-stick material such as teflon (PTFE), any of a variety of photoresists such as SU8 or any other thick film photoresist, or any combination of these materials.

In some embodiments, all or a portion of the microfluidic device (e.g., the cover or lid) may be fabricated from an optically transparent material to facilitate observation and monitoring of cells or objects entrapped within the device. In some embodiments, the different layers in a microfluidic device comprising multiple layers may be fabricated from different materials, e.g., a fluid channel layer may be fabricated from an elastomeric material while the device substrate and a cover plate may be fabricated from glass or another suitable material.

In some embodiments, the microfluidic device may comprise a three layer structure that includes a substrate, a fluid channel layer comprising a plurality of weir-traps, and a cover plate, whereby the volume of the microfluidic chambers (i.e., the interior regions of the traps) is determined by the cross-sectional area of the chambers and the thickness of the fluid channel layer. In some embodiments, the microfluidic device may comprise two layers, three layers, four layers, five layers, or more than five layers in total.

As indicated above, in some embodiments the thickness of a fluid channel layer will determine the depth of the fluid channels and microfluidic chambers (e.g., “micro-chambers”, “trapping chambers”, or the interior regions of the traps) within the device, and will thus influence the volume of the trapping chambers. In some embodiments, e.g., where fluid channels and trapping features are etched, embossed, or ablated into a substrate, the depth of the fluid channels and trapping chambers within the device will determined by the etch depth, embossed depth, or ablation depth, and will thus influence the volume of the trapping chambers. In some embodiments, e.g., where fluid channels and trapping features are etched, embossed, or ablated into a substrate, the fluid channels and trapping chambers may have the same depth or different depths.

In general, the depth of fluid channels and/or trapping chambers within the disclosed devices may range from about 1 μm and about 1 mm. In some embodiments, the depth of the fluid channels and/or trapping chambers may be at least 1 μm, at least 5 μm, at least 10 μm, at least 20 μm, at least 30 μm, at least 40 μm, at least 50 μm, at least 100 μm, at least 200 μm, at least 300 μm, at least 400 μm, at least 500 μm, at least 600 μm, at least 700 μm, at least 800 μm, at least 900 μm, or at least 1 mm. In some embodiments, the depth of the fluid channels and/or trapping chambers may be at most 1 mm, at most 900 μm, at most 800 μm, at most 700 μm, at most 600 μm, at most 500 μm, at most 400 μm, at most 300 μm, at most 200 μm, at most 100 μm, at most 50 μm, at most 40 μm, at most 30 μm, at most 20 μm, at most 10 μm, at most 5 μm, or at most 1 μm. Any of the lower and upper values described in this paragraph may be combined to form a range included within the disclosure, for example, the depth of the fluid channels and/or trapping chambers may range from about 50 μm to about 100 μm. Those of skill in the art will recognize that depth of the fluid channels and/or trapping chambers may have any value within this range, for example, about 95 μm.

In general, the dimensions of fluid channels and microfluidic chambers in the disclosed device designs will be optimized to (i) provide uniform and efficient delivery and trapping of cells or other objects suspended in a fluid passed through the device, and (ii) to minimize cell sample and/or assay reagent consumption. In general, the width of fluid channels or microfluidic chambers may be between about 10 μm and about 2 mm. In some embodiments, the width of fluid channels or microfluidic chambers may be at least 10 μm, at least 25 μm, at least 50 μm at least 100 μm, at least 200 μm, at least 300 μm, at least 400 μm, at least 500 μm, at least 750 μm, at least 1 mm, at least 1.5 mm, or at least 2 mm. In other embodiments, the width of fluid channels or microfluidic chambers may at most 2 mm, at most 1.5 mm, at most 1 mm, at most 750 μm, at most 500 μm, at most 400 μm, at most 300 μm, at most 200 μm, at most 100 μm, at most 50 μm, at most 25 μm, or at most 10 μm. Any of the lower and upper values described in this paragraph may be combined to form a range included within the disclosure, for example, the width of the fluid channels may range from about 100 μm to about 1 mm. Those of skill in the art will recognize that the width of the fluid channel may have any value within this range, for example, about 80 μm.

In general, the volumes of the microfluidic chambers (e.g., trapping chambers) used in the disclosed devices may range from about 1,000 μm³ to about 1 mm³. In some embodiments, the microfluidic chamber volume may be at least 1,000 μm³, at least 10,000 μm³, at least 100,000 μm³, at least 1,000,000 μm³, at least 0.2 mm³, at least 0.5 mm³, or at least 1 mm³. In some embodiments, the microfluidic chamber volume is at most 1 mm³, at most 0.5 mm³, at most 0.2 mm³, at most 1,000,000 μm³, at most 100,000 μm³, at most 10,000 μm³, or at most 1,000 μm³. Any of the lower and upper values described in this paragraph may be combined to form a range included within the disclosure, for example, the microfluidic chamber volume may range from about 100,000 μm³ to about 0.2 mm³. Those of skill in the art will recognize that the chamber volume may have any value within this range, for example, about 8,000 μm³.

In some embodiments, the number of weir-traps and/or microfluidic chambers in the plurality of traps and/or chambers contained within a device of the present disclosure may range from about 1 to about 10⁶, or more. In some embodiments, the number of traps and/or chambers within the device may be at least 1, at least 10, at least 100, at least 1,000, at least 10⁴, at least 10⁵, or at least 10⁶. In some embodiments, the number of traps and/or chambers within the device may be at most 10⁶, at most 10⁵, at most 10⁴, at most 1,000, at most 100, or at most 1. Any of the lower and upper values described in this paragraph may be combined to form a range included within the disclosure, for example, the number of traps and/or chambers within the device may range from about 100 to about 10,000. Those of skill in the art will recognize that the number of traps and/or chambers within the device may have any value within this range, for example, about 1,200.

In some embodiments, the pitch (or spacing) between weir-traps may range from about 100 μm to about 1,000 μm, or more. In some embodiments, the pitch between weir-traps may be at least at least 100 μm, at least 200 μm, at least 300 μm, at least 400 μm, at least 500 μm, at least 600 μm, at least 700 μm, at least 800 μm, at least 900 μm, or at least 1,000 μm. In some embodiments, the pitch between weir-traps may be at most 1,000 μm, at most 900 μm, at most 800 μm, at most 700 μm, at most 600 μm, at most 500 μm, at most 400 μm, at most 300 μm, at most 200 μm, or at most 100 μm. Any of the lower and upper values described in this paragraph may be combined to form a range included within the disclosure, for example, the pitch between weir-traps may range from about 200 μm to about 400 μm. Those of skill in the art will recognize that the pitch between weir-traps may have any value within this range, for example, about 220 μm.

If fabricated as a set of separate parts, the disclosed microfluidic devices may be assembled mechanically, e.g. by clamping two or more parts together (with or without the use of a gasket) using an appropriate fixture and fasteners, or parts may be assembled and bonded together using any of a variety of techniques (depending on the choice of materials used) known to those of skill in the art, for example, through the use of anodic bonding, thermal bonding, or any of a variety of adhesives or adhesive films, including epoxy-based, acrylic-based, silicone-based, UV curable, polyurethane-based, or cyanoacrylate-based adhesives.

Microfluidic devices comprising pumps or valves: In many embodiments, the disclosed microfluidic devices may be used with external pumps for controlling fluid flow through the device. In some embodiments, the disclosed microfluidic devices may further comprise active fluidic components such as pumps (e.g. micro-pumps) or valves (e.g. micro-valves) to provide additional control of fluid flow, e.g. to enable addressable control of fluid delivery to specific fluid compartments and/or to enable isolation of cells, beads, or other objects within specific fluid compartments. In some embodiments, one or more micropumps or microvalves may be fabricated within or directly integrated with the microfluidic device itself (e.g., in embodiments where the microfluidic device also comprises pre-packaged assay buffers, assay reagents, capture antibodies or capture probes conjugated to magnetic beads, and the like, or other fluids used in the operation of the device). In some embodiments, as noted above, one or more conventional pumps or valves may reside externally to the device, e.g. as a component included in an instrument module with which the microfluidic device interfaces, and be connected to the device via appropriate tubing. Examples of suitable micro-pumps (or fluid actuation mechanisms) for use in the devices of the present disclosure include, but are not limited to, electromechanically- or pneumatically-actuated miniature syringe or plunger mechanisms, membrane diaphragm pumps actuated pneumatically or by an external piston, pneumatically-actuated reagent and buffer pouches or bladders, or electro-osmotic pumps. Examples of suitable micro-valves for use in the devices of the present disclosure include, but are not limited to, pinch valves constructed using a deformable membrane or tube and pneumatic, magnetic, electromagnetic, or electromechanical (solenoid) actuation, one-way valves constructed using deformable membrane flaps, miniature check valves and gate valves; one-shot “valves” fabricated using wax or polymer plugs that can be melted or dissolved, or polymer membranes that can be punctured, and the like. In some embodiments of the disclosed microfluidic devices, each micro-chamber in a plurality of micro-chambers within the device will be individually addressable and isolatable by means of one or more micro-valves positioned at the inlet(s) and/or outlet(s) of each micro-chamber, thereby allowing the individual micro-chambers to be reversibly sealed in an addressable manner. In some embodiments, one or more subsets of a plurality of the micro-chambers will be addressable and isolatable as groups by means of one or more micro-valves positioned at common inlet(s) and/or outlet(s) for the one or more subsets. In some embodiments, the inlets and outlets of the device, or fluid channels therein, may include integrated check valves for controlling the directionality of fluid flow.

Microfluidic devices comprising sensors: In some embodiments, the microfluidic devices of the present disclosure, or one or more individual chambers of the plurality of chambers contained therein, may further comprise one or more additional components for use in regulating the microenvironment of cells or other objects within the device and maintaining cell viability. Examples include, but are not limited to, heating elements, cooling elements, temperature sensors, pH sensors, gas sensors (e.g., O₂ sensors, CO₂ sensors), electrodes, etc., or any combination thereof. In some embodiments, the microfluidic devices of the present disclosure may further comprise additional components or features, e.g., transparent optical windows to facilitate microscopic observation, microscopic imaging, and/or spectroscopic monitoring techniques; inlet and outlet ports for making connections to perfusion systems, electrical connections for connecting electrodes or sensors to external processors or power supplies, etc.

Compartmentalization of cells and/or beads within microfluidic devices: For some of the single cell analysis methods to be discussed in more detail below, it may be desirable to compartmentalize cells once they have been trapped by the array of trapping features within disclosed devices. Methods are disclosed herein for trapping cells, beads, or other objects within the entrance constrictions of all or a portion of the weir-traps within a device using a first, relatively low hydrodynamic pressure, and subsequently forcing the cells, beads, or other objects (provided that they are at least somewhat deformable) through the entrance constriction and into an interior region (or chamber) of the trap using a pulse of higher hydrodynamic pressure. The magnitude of the pressure required to force the cells, beads, or other objects through the entrance constrictions of the traps may vary depending on a variety of experimental parameters including, but not limited to, the type of cell, the growth stage (i.e., cell cycle stage) of the cell, the type of bead (size and composition), the dimensions of the constriction, the fluidic layout of the cell trapping device, etc. Examples of suitable devices for use with this method are shown in FIGS. 2A and 2B, FIGS. 4A and 4B, and FIGS. 6-10. In some instances, the weir-trap design used may comprise a frit within the exit region of the trap to facilitate containment of the trapped cell or object within the interior region of the trap, where the frit structure comprises one or more constrictions that have a spatial dimension that is smaller than the smallest dimension of the trapped cell or object.

In some embodiments of the disclosed methods, the first hydrodynamic pressure (or trapping pressure) may range from about 1 mbar to about 200 mbar. In some embodiments, the first hydrodynamic pressure (or trapping pressure) may be at least 1 mbar, at least 5 mbar, at least 10 mbar, at least 20 mbar, at least 30 mbar, at least 40 mbar, at least 50 mbar, at least 60 mbar, at least 70 mbar, at least 80 mbar, at least 90 mbar, at least 100 mbar, at least 150 mbar, or at least 200 mbar. In some embodiments, the first hydrodynamic pressure (or trapping pressure) may be at most 200 mbar, at most 150 mbar, at most 100 mbar, at most 90 mbar, at most 80 mbar, at most 70 mbar, at most 60 mbar, at most 50 mbar, at most 40 mbar, at most 30 mbar, at most 20 mbar, at most 10 mbar, at most 5 mbar, or at most 1 mbar. Any of the lower and upper values described in this paragraph may be combined to form a range included within the disclosure, for example, in some embodiments the first hydrodynamic pressure may range from about 10 mbar to about 80 mbar. Those of skill in the art will recognize that the first hydrodynamic pressure may have any value within this range, for example, about 92 mbar.

In some embodiments of the disclosed methods, the second hydrodynamic pressure (or compartmentalization pressure) may range from about 50 mbar to about 1,000 mbar. In some embodiments, the second hydrodynamic pressure (or compartmentalization pressure) may be at least 50 mbar, at least 100 mbar, at least 200 mbar, at least 300 mbar, at least 400 mbar, at least 500 mbar, at least 600 mbar, at least 700 mbar, at least 800 mbar, at least 900 mbar, or at least 1,000 mbar. In some embodiments, the second hydrodynamic pressure (or compartmentalization pressure) may be at most 1,000 mbar, at most 900 mbar, at most 800 mbar, at most 700 mbar, at most 600 mbar, at most 500 mbar, at most 400 mbar, at most 300 mbar, at most 200 mbar, at most 100 mbar, or at most 50 mbar. Any of the lower and upper values described in this paragraph may be combined to form a range included within the disclosure, for example, in some embodiments the second hydrodynamic pressure may range from about 200 mbar to about 800 mbar. Those of skill in the art will recognize that the first hydrodynamic pressure may have any value within this range, for example, about 860 mbar.

In some embodiments of the disclosed methods, the ratio of the second hydrodynamic pressure (or compartmentalization pressure) to the first hydrodynamic pressure (or trapping pressure) may range from about 5× to about 20×. In some embodiments, the ratio of second-to-first hydrodynamic pressures may be at least 5×, at least 10×, at least 12×, at least 14×, at least 16×, at least 18×, or at least 20×. In some embodiments, the ratio of second-to-first hydrodynamic pressure may be at most 20×, at most 18×, at most 16×, at most 14×, at most 12×, at most 10×, or at most 5×. Any of the lower and upper values described in this paragraph may be combined to form a range included within the disclosure, for example, in some embodiments the ratio of second-to-first hydrodynamic pressures may range from about 12× to about 16×. Those of skill in the art will recognize that the ratio of second-to-first hydrodynamic pressures may have any value within this range, for example, about 13.5×.

In some embodiments, the disclosed methods for trapping and compartmentalizing cells, beads, or other objects may be repeated at least once, twice, three times, four times, or more, thereby allowing two or more cells, beads, or objects to be confined within the interior region(s) of one or more weir-traps. In some instances, the low pressure trapping and high pressure compartmentalization steps are repeated at least once using a fluid that comprises the same cells, beads, or other objects as that used the first time. In some instances, the low pressure trapping and high pressure compartmentalization steps are repeated at least once using a fluid that comprises different cells, beads, or other objects than that used the first time, such that the at least two objects confined within the interior region(s) of one or more weir-traps comprise at least two of the same cells, at least two different cells, at least two of the same beads, at least two different beads, or at least one cell and one bead, or any other combination of cells, beads, or other objects.

Culturing cells within microfluidic devices: In some embodiments, the disclosed methods, devices, and systems may be used to culture single cells (or groups of cells) once thay have been trapped and compartmentalized within all or a portion of the weir-traps within a device. For example, following the trapping and compartmentalization steps, the inlet of the microfluidic device may be connected to a perfusion system which continuously or periodically supplies the compartmentalized cells with a supply of growth medium while a specified temperature is maintained using integrated or external heating/cooling mechanisms, and temperature, pH, O₂ concentration, CO₂ concentration, etc., may be monitored using integrated or external sensors.

In some instances, trapped and compartmentalized cells may be cultured within the disclosed devices for periods of time ranging from a day to several months. In some instances, the cells within the device may be cultured for at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 4 months, at least 5 months, or at least 6 months. In some instances, the cells within the device may be cultured for at most 6 months, at most 5 months, at most 4 months, at most 3 months, at most 2 months, at most 1 month, at most 3 weeks, at most 2 weeks, at most 1 week, at most 6 days, at most 5 days, at most 4 days, at most 3 days, at most 2 days, or at most 1 day. Any of the lower and upper values described in this paragraph may be combined to form a range included within the disclosure, for example, in some embodiments the cells with the device may be cultured for a period of time ranging from 1 week to 1 month. Those of skill in the art will recognize that the cells with the device may be cultured for a period of time having any value within this range, for example, about 2.5 weeks.

Isolation of cells and/or beads within microfluidic devices: For some of the single cell analysis methods to be discussed in more detail below, it may be desirable to both compartmentalize and isolate cells once they have been trapped by the array of trapping features within disclosed devices. Thus, methods are also disclosed herein for isolating individual cells, beads, or other objects, or combinations thereof, once they have been trapped, compartmentalized, and/or cultured within the entrance regions or within the interior regions of all or a portion of the weir-traps within a device. For example, in some instances, the weir-traps (or their interior regions) may be sealed by flowing an immiscible fluid through the device to prevent diffusion or mixing of components that have been released upon lysis of isolated cells. In some instances, the immiscible fluid may comprise oil. In some instances, the immiscible fluid may comprise air.

Use of immiscible fluids: The isolation of trapped and/or compartmentalized cells using an immiscible fluid such as oil is enabled due to the fact that the pressure required to force fluid to now through a microfluidic channel is dominated by the smallest dimension of the channel. Smaller dimensions require higher pressures to induce fluid flow. Furthermore, since the microfluidic channels within the disclosed devices are typically hydrophilic, an even larger external pressure is required to force hydrophobic oil into the channels. Oil will flow when the external pressure exceeds the critical value required to overcome capillary pressure:

Pₑₓₜ=γ(w⁻¹+h⁻¹)

where γ is the surface tension of the oil/water interface, and w and h are the width and height of the fluid channel. Assuming γ ˜50 mJ/m² for an oil/water interface, and that the width and height of the bypass channels have dimensions of 25 μm and 20 μm respectively, the critical pressure required to induce flow in the bypass channels is about 4.5 kPa (45 mbar). Conversely, the critical pressure to induce flow through a 5 μm constriction of the fluidic traps is about 12.5 kPa (125 mbar). This indicates that the optimal pressure to seal the micro-wells in oil is in the range of 5-10 kPa (50-100 mbar) for a device similar to that shown in FIG. 6. Air sealing may use even higher pressure differentials. Thus, one may identify a range of device design-dependent pressures in which an immiscible fluid, such as oil or air, will flow through the bypass channels but not through the weir-traps. This process allows each compartmentalized cell or group of cells trapped within the device to be sealed in an aqueous droplet surrounded by an oil or air interface.

The ability to tune the hydrodynamic resistance of the microfluidic device to achieve high flow rate for hydrophilic fluids through the trapping features, thereby allowing cells or other objects to be trapped at high efficiency within in the microfluidic constrictions, while preventing flow of hydrophobic fluids, thereby allowing the trapped cells to be isolated by a medium which prevents mixing and contamination and enables efficient techniques for massively parallel preparation of, for example, single cell cDNA libraries, constitutes a novel feature of the present disclosure. The combination of high trapping efficiency devices and cell isolation and barcoding methods (the latter to be discussed below) disclosed herein overcomes problems with poor cell trapping efficiency for single cell analysis techniques that rely on random Poisson statistics, such as sedimentation into micro-wells or encapsulation into water droplets surrounded by oil. The presently disclosed methods and devices also overcome problems with existing low-throughput technologies that are based on cell sorting using a flow cytometer, or existing microfluidic-based single cell trapping and barcoding approaches, such as the system produced by Fluidigm Corp. (South San Francisco, Calif.) which uses pumps and valves to deliver the barcodes dispersed in a fluid phase to each cell trap.

Use of hydrogels: Another method disclosed herein for isolating individual cells, beads, or other objects, or combinations thereof, once they have been trapped, compartmentalized, and/or cultured within the entrance regions or within the interior regions of all or a portion of the weir-traps within a device comprises the use of a semipermeable, biocompatible hydrogel. In some instances, the disclosed microfluidic devices may comprise a removable lid which is mechanically clamped or otherwise adhered to the fluid channel layer of the device (i.e., with sufficient force to withstand the moderate hydrodynamic pressures required for introducing cells or other objects into the array of weir-traps within the device). Cells, beads, or other objects that have been trapped or compartmentalized within the disclosed devices can then be sealed in the semipermeable hydrogel, e.g., by flowing in cross-linkable solution through the device that is subsequently polymerized to transform the fluidic layer into a hydrogel, after which the microfluidic device lid can be removed to allow access to the trapped cells, beads, or other objects. Examples of gels that can be used include, but are not limited to, polyethylene glycol gels, hyaluronic acid gels, gelatin methacrylates, UV curable gels, thiol-crosslinkable gels, alginate gels, agarose gels, etc.

A significant advantage of this approach is the ability to exploit the semi-permeable nature of the hydrogel, which allows for fast diffusion of small molecules (e.g., short DNA or RNA strands, lysis chemicals, enzymes, and other reverse transcription reagents), while hindering the diffusion of long DNA or RNA molecules, viral particles, large proteins, antibodies, or other large macromolecules. This feature allows, for example, a cell lysate to remain trapped inside the hydrogel during subsequent DNA barcoding steps as will be discussed in more detail below, thereby allowing cellular components to be associated with a unique molecular identifier that can be traced back to a specific individual cell during subsequent nucleic acid sequencing analysis. In some instances, the ability to remove the lid of the device and directly access the cells (or other objects) immobilized within the hydrogel allows one to print DNA barcodes, cell lysis buffers, and/or other reagents (e.g., using inkjet printing or dip-pen nanolithography techniques) to specific cells after the cells have been introduced and sealed in the hydrogel.

Molecular barcoding of single cells and cellular components: Also disclosed herein are methods for using the disclosed high efficiency cell trapping devices for molecular barcoding of cellular components derived from single cells, e.g., using the methodology described by Fan, et al. (2015), “Combinatorial labeling of single cells for gene expression cytometry”, Science 347(6222): 1258367. There are several existing approaches for compartmentalizing single cell lysates along with DNA barcodes inside aqueous droplets that are encapsulated in oil. These are mostly based on random distributions of cells and barcode molecules based on Poisson statistics, in which cells and barcodes are randomly encapsulated in oil/water droplets (known as Drop-Seq and its variations), or in which cells and barcodes are randomly deposited on microfluidic templates and sealed in oil (known as Seq-Well and its variations). Neither of these approaches is able to determine a priori which drop (or micro-well) contains which DNA barcode, thus these techniques are unable to link image-based phenotypic data to the genomic data for each cell.

There are other platforms which intentionally place DNA barcodes at known locations, such as the WaferGen (Fremont, Calif.) platform, which deposits a unique DNA barcode at the bottom of an array of micro-wells machined in an aluminum plate, and also the Becton Dickinson Resolve™ platform, which deposits a unique DNA barcode at the bottom of each well in 96- or 384-well microtiter plates and then sorts single cells into each well. Finally, as noted above, the Fluidigm platform uses pneumatic pumps to deliver DNA barcodes in a fluid dispersion into each microfluidic trap. However, these systems are unable to achieve the trapped cell density of Poisson-based approaches, or that of the presently disclosed methods and devices.

Disclosed herein are methods for organizing arrays of single cells within the novel microfluidic devices described above which may comprise: (1) flowing cells in an aqueous suspension through a microfluidic device that comprises an array of trapping features and interconnecting bypass channels, thereby allowing single cells to be trapped within the array, (2) replacing the fluid with lysis buffers and other biochemical reagents, (3) isolating the trapped cells or cell lysates by flowing an immiscible fluid such as oil or air through the microfluidic device, and (4) attaching cell lysate components to unique molecular barcodes that allow them to be traced after they are pooled and analyzed using conventional or next generation sequencing techniques, or any combination thereof. Any of a variety of cell lysis techniques known to those of skill in the art may be used, as will be discussed in more detail below.

In some instances, the unique molecular barcodes comprise patterned DNA barcodes that will allow for both image-based phenotyping of the trapped cells, and then molecular transcript-based genotyping of each trapped cell by converting the mRNA of the single cell lysate into cDNA, which are appended to the DNA barcodes in each trap.

In some instances, multiple copies of a unique molecular barcode may be synthesized in situ within each weir-trap of the device (e.g., using light-directed synthesis techniques such as those described by Fodor, et al. (1991), “Light-directed, spatially addressable parallel chemical synthesis”, Science 251(4995):767-773 or McGall, et al. (1996), “Light-directed synthesis of high-density oligonucleotide arrays using semiconductor photoresists”, Proc. Natl. Acad. Sci. USA 93(24): 13555-13560) prior to assembly of the device and prior to its use in cell trapping. In some instances the unique molecular barcodes, e.g., oligonucleotide barcodes, that are synthesized within each weir-trap may be covalently tethered to a surface within the weir-trap (e.g., a substrate surface within the interior region of a weir-trap) using any of a variety of photo-cleavable or chemically-cleavable linkers known to those of skill in the art.

In some instances, multiple copies of a unique molecular barcode may be printed into each weir-trap of the device (e.g., using ink-jet printing or dip-pen nanolithography techniques) prior to assembly of the device and prior to its use in cell trapping. In some instances, the unique molecular barcodes (e.g., oligonucleotide barcodes) that are printed into each weir-trap may be either non-specifically adsorbed to a surface within the weir-trap, or may be covalently tethered to a surface with the weir-trap using any of a variety of photo-cleavable or chemically-cleavable linkers known to those of skill in the art.

The disclosed methods include methods for printing or synthesizing DNA barcodes directly within the microfluidic device prior to introducing the cells, as well as methods to print DNA barcodes, cell lysis buffers, and/or other reagents to specific cells after the cells have been introduced and sealed in a hydrogel. The advantage of the first technique (printing barcodes prior to cell organization) is that each cell will have a unique barcode already positioned in the correct location. The advantage of the second technique (printing barcodes after cell organization) is that it is possible to limit the DNA barcoding to those cells which exhibit interesting cell phenotypes, which may be identified, e.g., through the use high content image-based assays.

In some instances, multiple copies of a unique molecular barcode may be tethered to each bead in a library of beads. Beads may be trapped and compartmentalized with cells within the disclosed devices, e.g., one bead per cell, so that upon lysis each component of the cell lysate may be tagged with a molecular barcode that identifies the cell of origin following downstream sequencing analysis. In some instances the beads may be magnetic beads.

In some instances, the molecular barcodes may comprise a target recognition sequence or element that hybridizes with or binds to a specific molecular component, e.g., through hybridization to the poly(A) tail of mRNA molecules. In some instances the barcodes may comprise an oligonucleotide barcode conjugated to an antibody or other molecular recognition element that binds specifically to an antigen or other molecular component.

In some instances, the unique molecular barcodes that encode the identity of an individual cell (total barcode library diversity on the order of ˜10⁶ or greater to ensure that each cell is paired with a unique barcode) may also comprise a molecular counter region comprising a diversity on the order of ˜10⁵ or greater so that each individual mRNA molecule (or other oligonucleotide target molecules, protein targets, etc., as defined by the target recognition portion of the molecular barcode) within a cell becomes specifically labeled and may be counted on the basis of it unique molecular counter. After cell lysis, performed for example by introducing a suitable lysis buffer to the array of trapping chambers, the released mRNA molecules (or other target molecules) hybridize to (or bind to) the molecular barcodes (which may remain tethered to a bead or to a surface within the trapping chamber, or which may have been released into solution), and subsequent reverse transcription, amplification, and/or sequencing reactions may be performed. In some instances, the molecular barcodes remain tethered to beads which are subsequently retrieved from the array and pooled for reverse transcription, amplification, and sequencing. For single cell gene expression profiling studies, complementary DNA strands (cDNAs) from all polyadenylated transcripts derived from each single cell are covalently archived on the surface of each single bead therefore any selection of genes can be analyzed. The gene expression profile for each cell is reconstructed when barcoded transcripts are assigned to the cell of origin and counted. In some embodiments, the reverse transcription reactions may be performed within the chambers of the cell trapping array, e.g., prior to retrieval of beads. In some embodiments, an amplification reaction (e.g. a PCR amplification or an isothermal amplification reaction) and/or sequencing reactions (e.g. cyclic sequencing by synthesis reactions) may also be performed within the chambers of the cell trapping array. In some embodiments, each individual chamber or bead within a plurality of chambers or beads may comprise two or more target molecule recognition sequences or elements. In some embodiments, two or more beads may be co-compartmentalized with each single cell within the array, wherein different beads comprise different target recognition sequences or target recognition elements that are directed to different oligonucleotide or protein target molecules (e.g. mRNA molecules, tRNA molecules, fragments of genomic DNA, specific receptor proteins or enzymes, and the like). Retrieval of the beads would then allow downstream processing of molecular barcodes for counting different types of target molecules associated with each single cell. In some embodiments, one or more molecular sensing beads, e.g., cytokine sensing beads, and molecular barcoding beads may be co-compartmentalized (simultaneously or sequentially) with single cells to monitor, e.g., cytokine secretion patterns or changes in cytokine secretion pattern following exposure to a chemical stimulus, followed by lysis of the cell and molecular barcoding of the released mRNA molecules to correlate changes in gene expression profile with changes in secretion patterns.

Also disclosed herein are methods for organizing arrays of single cells within the novel microfluidic devices described above which may comprise: (1) flowing cells in an aqueous suspension through a microfluidic device that comprises an array of trapping features and interconnecting bypass channels, thereby allowing single cells to be trapped within the array, (2) isolating the trapped cells within the array by flowing a cross-linkable solution through the microfluidic device that transforms the fluidic layer into a hydrogel, (3) removing the lid of the microfluidic device to enable direct access to the array of cells (or other objects) trapped in the hydrogel, and (4) attaching the cell lysates to unique barcodes that allow them to be traced after they are pooled and analyzed using conventional or next generation sequencing techniques, or any combination thereof. This approach allows one to leverage the native porosity of the hydrogel to trap large molecules, like mRNA transcripts, long DNA strands, large proteins, virus particles, etc., while allowing passage of small molecules into the gel, including lysis reagents, enzymes, short DNA strands (e.g., those shorter than 100-200 bp), and other reagents typically used in molecular biology protocols.

As noted above, any of a variety of techniques may be used to perform lysis of cells once they have been trapped. Examples include, but are not limited to, the use of heat, acoustic power, optical laser pulses, electric field pulses, freeze/thaw cycles, and chemical reagents. In some instances cell lysis can be achieved by injecting lysis buffer in an aqueous solution prior to oil- or air-sealing of the trapping chambers, or by dissolving a lysis buffer into an oil-sealing medium.

We have also demonstrated that the trapping chambers within the device can be unsealed at the end of an experiment which allows, for example, beads or barcoded cDNA to be retrieved from the cell trapping device.

Any of a variety of nucleic acid sequencing methods and platforms known to those of skill in the art may be used with the molecular barcoding methods disclosed herein. Examples include, but are not limited to, paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof.

In some embodiments, high-throughput sequencing methods, such as cyclic array sequencing using platforms such as Roche 454, Illumina Solexa, ABI-SOLiD, ION Torrent, Complete Genomics, Pacific Bioscience, Helicos, or the Polonator platform, may be utilized. In some embodiments, sequencing may comprise the use of an Illumina MiSeq, HiSeq, or other sequencing platform. In some embodiments, sequencing may comprise the use of the Minion or related sequencing devices being commercialized by Oxford Nanopore, the use of the Genius system from Genapsys, or the Hyb & SeC™ single molecule, direct digital sequencing technology from Nanostring. In some embodiments, sequencing may comprise the use of digital spatial profiling (DSP) technology such as that available from Nanostring.

Imaging-based phenotypic analysis & correlation with genomic data: The disclosed microfluidic devices for trapping of cells and other objects are designed to facilitate high resolution, imaging-based analysis of cell phenotypic traits, which in some instances may then be correlated with genomic data obtained as discussed above.

Any of a variety of imaging techniques known to those of skill in the art may be employed in performing phenotypic analysis of cells trapped within the disclosed microfluidic devices. Examples include, but are not limited to, bright-field imaging, dark-field imaging, fluorescence imaging, luminescence imaging, chemiluminescence imaging, phosphorescence imaging, phase-contrast imaging, quantitative phase contrast imaging, confocal microscopy imaging, super resolution microscopy imaging, or time-resolved fluorescence imaging. In some embodiments, dual wavelength excitation and emission (or multi-wavelength excitation or emission) fluorescence imaging may be performed. In some embodiments, two-photon fluorescence imaging may be performed. In some embodiments, coherent Raman imaging may be performed.

In some instances, a series of one or more images acquired using a high-throughput microscopy imaging system may be pre-processed to, for example, correct image contrast and brightness, correct for non-uniform illumination, correct for an optical aberration (e.g., a spherical aberration, a chromatic aberration, etc.), remove noise, identify objects (e.g., cells or sub-cellular structures) within each of the images, segment each of the images to isolate the identified objects, tile segmented images to create composite images, perform feature extraction (e.g., identification and/or quantitation of object properties such as observable cellular phenotypic traits), or any combination thereof. In some instances, a plurality of chambers within the device may be imaged within a single image. In some instances, a series of images may be “tiled” to create a high resolution image of all or a portion of the plurality of chambers within the device.

In some instances, automated or semi-automated image processing may be utilized to identify and count cells or beads within trapping chambers, monitor cells or beads within trapping chambers to identify specified subsets of cells or beads, e.g., dead cells, live cells, pairs of cells, cells that are actively dividing, cells exhibiting specific cell surface markers, internal cellular proteins labeled with fluorescent markers, fluorescent chemical sensing beads, etc. Examples of image processing algorithms that may be used in implementing the disclosed methods include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation algorithms (e.g., intensity thresholding, intensity clustering methods, intensity histogram-based methods, etc.), feature and pattern recognition algorithms (e.g., the generalized Hough transform for detecting arbitrary shapes, the circular Hough transform, etc.), and mathematical analysis algorithms (e.g., Fourier transform, fast Fourier transform, wavelet analysis, auto-correlation, etc.), or any combination thereof.

Machine learning-based image processing for cell phenotyping: In some preferred embodiments, machine learning-based approaches may be used to implement all or a portion of the disclosed methods for detection and counting of individual cells, analysis of cell phenotypic traits, and correlation with genomic data. In some instances, machine learning-based approaches to image processing may be used, for example, to automatically align and crop images of individual trapping chambers with a microfluidic device from larger images, determine the specific row and column addresses of each chamber, identify each cell in a chamber at each time point, analyze the fluorescent signature of each cell to determine the presence of reporter genes, proteins, or other molecular features, and/or then plot the number of cells within each chamber or colony (optionally along with a list of their molecular features). In some instances, machine learning-based image processing may be used to classify cells based on their phenotypic traits according to a pre-specified set of classification criteria. In some instances, machine learning-based image processing may be used to classify cells based on their phenotypic traits according to a set of classification criteria derived by the machine learning algorithm.

Any of a variety of machine learning algorithms known to those of skill in the art may be suitable for use in the disclosed methods. Examples include, but are not limited to, supervised learning algorithms, unsupervised learning algorithms, semi-supervised learning algorithms, reinforcement learning algorithms, deep learning algorithms, or any combination thereof.

Supervised learning algorithms: In the context of the present disclosure, supervised learning algorithms are algorithms that rely on the use of a set of labeled training data (e.g., cell phenotypic traits and the corresponding known cell classification types) to infer the relationship between the set of phenotypic traits for a given cell or cell sample and a classification of the cell or cell sample. The training data comprises a set of paired training examples, e.g., where each example comprises a set of phenotypic trait data and the resultant classification of the given cell according to conventional methods.

Unsupervised learning algorithms: In the context of the present disclosure, unsupervised learning algorithms are algorithms used to draw inferences from training datasets consisting of cell phenotypic trait datasets that are not paired with labeled cell classification data. The most commonly used unsupervised learning algorithm is cluster analysis, which is often used for exploratory data analysis to find hidden patterns or groupings in process data.

Semi-supervised learning algorithms: In the context of the present disclosure, semi-supervised learning algorithms are algorithms that make use of both labeled and unlabeled cell classification data for training (typically using a relatively small amount of labeled data with a large amount of unlabeled data).

Reinforcement learning algorithms: In the context of the present disclosure, reinforcement learning algorithms are algorithms which are used, for example, to determine a set of cell phenotypic data processing steps that should be taken so as to maximize a cell classification reward function. Reinforcement learning algorithms are commonly used for optimizing Markov decision processes (i.e., mathematical models used for studying a wide range of optimization problems where future behavior cannot be accurately predicted from past behavior alone, but rather also depends on random chance or probability). Q-learning is an example of a class of reinforcement learning algorithms. Reinforcement learning algorithms differ from supervised learning algorithms in that correct training data input/output pairs are never presented, nor are sub-optimal actions explicitly corrected. These algorithms tend to be implemented with a focus on real-time performance through finding a balance between exploration of possible outcomes based on updated input data and exploitation of past training.

Deep learning algorithms: In the context of the present disclosure, deep learning algorithms are algorithms inspired by the structure and function of the human brain called artificial neural networks (ANNs), and specifically large neural networks comprising multiple hidden layers, that are used to map an input data set (e.g. a cell phenotypic trait data set) to an output (e.g., cell type) classification decision. Artificial neural networks will be discussed in more detail below.

Artificial neural networks & deep learning algorithms: In one preferred embodiment, the machine learning algorithm employed in the disclosed methods may be an artificial neural network (ANN) or deep learning algorithm. One or more of the image processing steps used in a conventional image processing approach may be augmented or replaced with the use of one or more artificial neural networks or deep learning algorithms. The artificial neural network may comprise any type of neural network model, such as a feedforward neural network, radial basis function network, recurrent neural network, or convolutional neural network, and the like. In some embodiments, the disclosed methods may employ a pre-trained ANN or deep learning architecture. In some embodiments, the disclosed methods may employ an ANN or deep learning architecture wherein the training data set is continuously updated with real-time cell classification data from a single local cell analysis system (i.e., a computer system or processor running a software program comprising the disclosed data processing methods), from a plurality of local cell analysis systems, or from a plurality of geographically-distributed cell analysis systems that are connected through the internet.

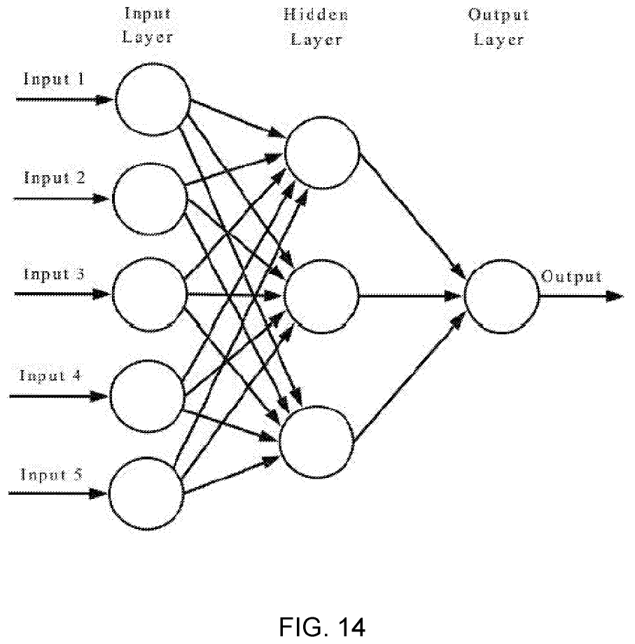

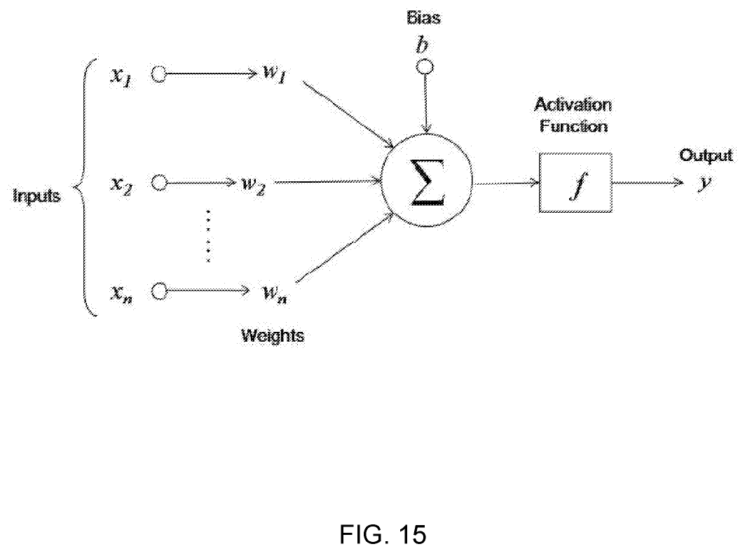

Artificial neural networks generally comprise an interconnected group of nodes organized into multiple layers of nodes (FIG. 14). For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm is an ANN comprising a plurality of hidden layers, e.g., two or more hidden layers. Each layer of the neural network comprises a number of nodes (or “neurons”). A node receives input that comes either directly from the input data (e.g., cell phenotype data) or the output of nodes in previous layers, and performs a specific operation, e.g., a summation operation. In some cases, a connection from an input to a node is associated with a weight (or weighting factor). In some cases, the node may sum up the products of all pairs of inputs, xi, and their associated weights (FIG. 15). In some cases, the weighted sum is offset with a bias, b, as illustrated in FIG. 15. In some cases, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sinc, Gaussian, or sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., a cell classification result) that the ANN computes are consistent with the examples included in the training data set. The parameters may be obtained from a back propagation neural network training process that may or may not be performed using the same computer system hardware as that used for performing the cell analysis methods disclosed herein.

Other specific types of deep machine learning algorithms, e.g., convolutional neural networks (CNNs) (e.g., often used for the processing of image data from machine vision systems) may also be used by the disclosed methods and systems. CNNs are commonly composed of layers of different types: convolution, pooling, upscaling, and fully-connected node layers. In some cases, an activation function such as rectified linear unit may be used in some of the layers. In the CNN architecture, there can be one or more layers for each type of operation performed. The CNN architecture may comprise any number of layers in total, and any number of layers for the different types of operations performed. The simplest convolutional neural network architecture starts with an input layer followed by a sequence of convolutional layers and pooling layers, where each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parametric rectified linear unit, exponential linear unit, softPlus, bent identity, softExponential, Sinusoid, Sinc, Gaussian, the sigmoid function and various others. The convolutional, pooling and ReLU layers may function as learnable features extractors, while the fully connected layers may function as a machine learning-based classifier.

As with other artificial neural networks, the convolutional layers and fully-connected layers of CNN architectures typically include various computational parameters, e.g., weights, bias values, and threshold values, that are trained in a training phase as described above.

In general, the number of nodes used in the input layer of the ANN (which determines the size of the input data set) may range from about 10 to about 100,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 5000, at least 10,000, at least 20,000, at least 30,000, at least 40,000, at least 50,000, at least 60,000, at least 70,000, at least 80,000, at least 90,000, or at least 100,000. In some instances, the number of node used in the input layer may be at most 100,000, at most 90,000, at most 80,000, at most 70,000, at most 60,000, at most 50,000, at most 40,000, at most 30,000, at most 20,000, at most 10,000, at most 5000, at most 4000, at most 3000, at most 2000, at most 1000, at most 900, at most 800, at most 700, at most 600, at most 500, at most 400, at most 300, at most 200, at most 100, at most 50, or at most 10. Any of the lower and upper values described in this paragraph may be combined to form a range included within the disclosure, for example, in some embodiments the number of nodes used in the input layer may range from 500 to 2,000. Those of skill in the art will recognize that the number of nodes used in the input layer may have any value within this range, for example, about 512 nodes.

In some instance, the total number of layers used in the ANN (including input and output layers) may range from about 3 to about 20. In some instance the total number of layer may be at least 3, at least 4, at least 5, at least 10, at least 15, or at least 20. In some instances, the total number of layers may be at most 20, at most 15, at most 10, at most 5, at most 4, or at most 3. Any of the lower and upper values described in this paragraph may be combined to form a range included within the disclosure, for example, in some embodiments the total number of layers may range from about 5 to about 15. Those of skill in the art will recognize that the total number of layers used in the ANN may have any value within this range, for example, 8 layers.

In some instances, the total number of learnable or trainable parameters, e.g., weighting factors, biases, or threshold values, used in the ANN may range from about 1 to about 10,000. In some instances, the total number of learnable parameters may be at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters may be any number less than 100, any number between 100 and 10,000, or a number greater than 10,000. In some instances, the total number of learnable parameters may be at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Any of the lower and upper values described in this paragraph may be combined to form a range included within the disclosure, for example, in some embodiments the total number of learnable or trainable parameters may range from about 100 to about 5,000. Those of skill in the art will recognize that the total number of learnable parameters used may have any value within this range, for example, about 2,200 parameters.

Training data sets: As noted above, the input data for training of the ANN or deep learning algorithm may comprise a variety of input values depending on which step(s) of the conventional image processing method are being replaced. In general, the input data for training of the ANN or deep learning algorithm will be data comprising the same set of input values, or a similar set of input values, as those used for determining a cell classification result for an actual test cell sample. Input data values may comprise numeric values (e.g., integer values, real values, floating point numbers, RGB or greyscale intensity values for individual pixels or binned pixels from an image), alphanumeric values, ascii values, etc., or any combination thereof. In general, the ANN or deep learning algorithm may be trained using one or more training data sets comprising the same or different sets of input (e.g., phenotypic trait) data and paired output (e.g., cell classification) data.

Instrument systems: Also disclosed herein are instrument systems that may comprise: a microfluidic cell trapping device as described herein, a light source, an image sensor, a fluid flow controller, a temperature controller, gas and pH controllers, and a processor, or any combination thereof.

Light sources: Any of a variety of light sources may be used to provide the excitation and/or imaging light, including but not limited to, tungsten lamps, tungsten-halogen lamps, arc lamps, lasers, light emitting diodes (LEDs), or laser diodes. In some instances, a combination of one or more light sources, and additional optical components, e.g. lenses, filters, apertures, diaphragms, mirrors, and the like, will comprise an illumination sub-system or module.

Image sensors: Any of a variety of image sensors may be used for imaging purposes, including but not limited to, photodiode arrays, charge-coupled device (CCD) cameras, or CMOS image sensors, micro-lens arrays, scanners, or other optical detection means. Imaging sensors may be one-dimensional (linear) or two-dimensional array sensors. In some instances, a combination of one or more image sensors, and additional optical components, e.g. lenses, filters, apertures, diaphragms, mirrors, and the like, will comprise an imaging sub-system or module.

The imaging module will often include a variety of optical components for steering, shaping, filtering, or focusing light beams. Examples of suitable optical components include, but are not limited to, lenses, mirrors, prisms, diffraction gratings, colored glass filters, narrowband interference filters, broadband interference filters, dichroic reflectors, optical fibers, optical waveguides, and the like. In some instances, the imaging module may further comprise one or more translation stages or other motion control mechanisms for the purpose of moving the microfluidic device relative to the illumination and/or imaging sub-systems, or vice versa.

Fluid flow controller: In some instances, the disclosed instrument systems (or cell analysis platforms) may comprise a fluid flow controller or perfusion system that provides programmable control of one or more fluid actuation mechanisms used to drive fluid flow in the microfluidic device. Examples of suitable fluid actuation mechanisms for use in the disclosed methods, devices, and systems include application of positive or negative pressure to fluid reservoirs connected to one or more device inlets or outlets, electrokinetic forces, electrowetting forces, passive capillary action, capillary action facilitated through the use of membranes and/or wicking pads, and the like.

Control of fluid flow through the disclosed microfluidic devices will often be performed through the use of one or more pumps (or other fluid actuation mechanisms) and one or more valves which, in some embodiments, will be housed externally to the device in a user-controlled instrument module. Examples of suitable pumps include, but are not limited to, syringe pumps, programmable syringe pumps, peristaltic pumps, diaphragm pumps, and the like. In some instances, fluid flow through the system may be controlled by means of applying positive pneumatic pressure at one or more inlets of external reagent and buffer containers connected to the microfluidic device, or at one or more inlets of the microfluidic device itself. In some instances, fluid flow through the device may be controlled by means of drawing a vacuum at one or more outlets of a waste reservoir connected to the device, or at the one or more outlets of the device. Examples of suitable valves include, but are not limited to, check valves, electromechanical two-way or three-way valves, pneumatic two-way and three-way valves, and the like.

Different fluid flow rates may be utilized at different points in the microfluidic device operating sequence. For example, in some instances of the disclosed methods, devices, and systems, the volumetric flow rate through all or a portion of the microfluidic device may vary from about −10 ml/sec to about +10 ml/sec. In some embodiments, the absolute value of the volumetric flow rate may be at least 0.00001 ml/sec, at least 0.0001 ml/sec, at least 0.001 ml/sec, at least 0.01 ml/sec, at least 0.1 ml/sec, at least 1 ml/sec, or at least 10 ml/sec, or more. In some embodiments, the absolute value of the volumetric flow rate may be at most 10 ml/sec, at most 1 ml/sec, at most 0.1 ml/sec, at most 0.01 ml/sec, at most 0.001 ml/sec, at most 0.0001 ml/sec, or at most 0.00001 ml/sec. The volumetric flow rate at a given point in time may have any value within this range, e.g. a forward flow rate of 1.2 ml/sec, a reverse flow rate of −0.07 ml/sec, or a value of 0 ml/sec (i.e. stopped flow).

In some embodiments, the disclosed cell analysis platforms may further comprise a temperature controller for maintaining a user-specified temperature within the microfluidic device, e.g., to enable cells to be incubated and maintained for extended periods while under continuous microscopic observation, or for ramping temperature between two or more specified temperatures over two or more specified time intervals. Examples of temperature control components that may be incorporated into the microfluidic device or into the instrument system include, but are not limited to, resistive heating elements (e.g. indium tin oxide resistive heating elements), Peltier heating or cooling devices, heat sinks, thermistors, thermocouples, infrared light sources, and the like, which are regulated using electronic feedback loops.

In some instances, the temperature controller may provide for a programmable temperature change at one or more specified, adjustable times prior to performing specific device operational steps. In some instances, the temperature controller may provide for programmable changes in temperature over specified time intervals. In some embodiments, the temperature controller may further provide for cycling of temperatures between two or more set temperatures with specified frequencies and ramp rates so that thermal cycling for amplification reactions may be performed.

Gas & pH controllers: In some embodiments, the disclosed cell analysis platforms may comprise gas and pH controllers and related components (e.g. sensors) for maintaining a user-specified percentage of gas, e.g. CO₂, or user-specified pH in buffers, growth media, or other fluids being delivered to the microfluidic device. Examples of suitable sensors include non-dispersive infrared (NDIR) CO₂ sensors (used in conjunction with an attenuated total internal reflection (ATR) optics for dissolved CO₂ sensing), metal insulator semiconductor field effect transistor (MOSFET)-type sensors for dissolved CO₂ sensing (e.g., having Pt—NiO thin films as the active CO₂ sensing material deposited on the gate electrode), CO₂-sensitive electrodes (e.g., Mettler Toledo's InPro 5000i dissolved CO₂ sensor series), pH-sensitive electrodes, pads immersed in the fluid, which produce a color change corresponding to the amount of dissolved CO₂ or the pH in the fluid such as those sold under the tradename Presens® sensor spots [PreSens Precision Sensing, GmbH, Regensburg, Germany], and the like. For control of CO₂ and pH, suitable sensors are used in a feedback loop to control acid/base titrations and CO₂ injection. In some embodiments, CO₂ or other gas concentrations, or pH, may be monitored directly in the fluid contained within the device. In some embodiments, CO₂ or other gas concentrations may be monitored in a gas or atmosphere which is in equilibrium with the fluid within the device.

Processors and computer systems: In many instances, the disclosed instrument systems (cell analysis platforms) will comprise a computer (or processor) and computer-readable media that includes code for providing a user interface as well as for manual, semi-automated, or fully-automated control of all system functions, e.g. control of the fluid flow control sub-system, the temperature and gas control sub-systems, the imaging subsystem, and the motion control sub-system if a translation stage is included. In many instances, the disclosed instrument systems will also comprise computer-readable media that includes code for performing conventional and/or machine learning-based image processing, as described above. In some embodiments, the system computer or processor may be an integrated component of the instrument system (e.g. a microprocessor, field programmable gate array (FPGA), or mother board embedded within the instrument). In some embodiments, the system computer or processor may be a stand-alone module, for example, a personal computer or laptop computer. In some instances, image data, sensor data, and/or other system data may be stored locally. In some instances, all or a portion of the image data, sensor data, and/or other system data may be stored in a cloud-based database. In some instances, all or a portion of the image processing may be performed locally or in the cloud.

Examples of fluid control functions provided by the instrument control software include, but are not limited to, volumetric fluid flow rates, fluid flow velocities, the timing and duration for introduction of cell sample(s) and/or bead samples, assay reagent addition, the delivery of chemical or physical stimuli, valve switching, and rinse steps.

Examples of temperature control functions provided by the instrument control software include, but are not limited to, specifying temperature set point(s) and control of the timing, duration, and ramp rates for temperature changes.

Examples of gas control functions provided by the instrument control software include, but are not limited to, control of CO₂ concentration.

Examples of imaging system control functions provided by the instrument control software include, but are not limited to, autofocus capability, control of illumination or excitation light exposure times and intensities, control of image acquisition rate, exposure time, and data storage options.

Examples of translation stage system control functions provided by the instrument control software include, but are not limited to, control of the stage position, orientation, and the timing and time duration thereof.

In some embodiments, the use of microfluidic control systems comprising multiple, independently-controllable flow channels and integrated fluidic valves may provide better control of the micro-environment of single cells within the trapping arrays, and enable one to control the timing and exposure level of the arrayed cells to different stimulatory compounds. In some embodiments, the cell analysis platform may utilize a microfabricated valve system to open and close microfluidic chambers, as needed, e.g., to control the exposure of bead-based sensing reagents to cell secretions.

Applications: The disclosed cell analysis platforms enable image-based phenotyping and molecular barcoding of single cells with a greater than 100-fold increase in throughput over flow-based sorting and existing fluidic trapping approaches. Furthermore, the imaging-based phenotyping capability of the disclosed systems cannot be implemented using Drop-Seq or other Poisson-based library preparation approaches. The disclosed cell analysis platforms are compatible with small samples, such as tumor biopsies, and may be used in future clinical applications for precision drug screening of tumor response. In some applications, the disclosed cell analysis platforms may be used for chromatin analysis.

In some instances, of the disclosed methods, devices, and systems, robust, massively parallel workflows for preparing single cell-based cDNA libraries is achieved, for example, by exposing single cells that have been immobilized in a hydrogel to lysis chemicals and reverse transcription reagents while relying on the small pore size of the hydrogel to locally confine the mRNA from single cell lysates, and then appending mRNA molecules to locally placed DNA barcodes. In some instances, the DNA barcodes may be printed or synthesized directly within the microfluidic device—one unique cell identification barcode per single cell trap—and the method relies on the ability of the mRNA molecules to diffuse over short distances towards a surface within the trap. In some instances, the DNA barcodes may be printed or synthesized directly within the microfluidic device prior to use and then released from a surface within the device, e.g., by cleaving a photo-cleavable or chemically-cleavable linker by which they were attached to the surface. In other instances, single cells may be trapped and immobilized within a hydrogel using a microfluidic device comprising a removable lid, and then DNA barcodes may be printed directly on the cells of interest which are identified through image-based phenotyping. In yet another application, single cells may be trapped and immobilized within a hydrogel, and upon removing the lid then exposed to a drug to identify phenotypic responses of single cells. Examples of phenotypic responses that may be observed include, but are not limited to, release of cytokines, shedding of viral particles, changes in growth cycle, or proliferation of cells. The small pore size of the hydrogel effectively immobilizes the cells and any large molecules that are secreted, while allowing one track phenotypic changes in each cell through time. This approach is applicable to both adherent and suspension cells. The use of molecular barcoding techniques in conjunction with the disclosed methods and devices allows one to correlate phenotypic traits, or changes thereof, to genomic data (e.g., changes in gene expression profiled) within single cells.

### EXAMPLES

These examples are provided for illustrative purposes only and not to limit the scope of the claims provided herein.

### Example 1—Prediction of Two Unique Flow Regimes in Mesh Fluid Networks

Hydrodynamic systems comprised of ladder and mesh networks can be modeled like electrical circuits, where the pressure, flow rate, and hydrodynamic resistances are analogous to voltage, current, and electrical resistances. As noted above, an example of a mesh-like fluidic network of the present disclosure is shown in FIGS. 4A and 4B. The equivalent resistance circuit is shown in FIG. 5

Mesh networks are comprised of two types of resistors, including those aligned parallel to the main flow path, i.e., R_{A} and R_{T}, and those aligned perpendicular to the main flow path, i.e., R_{B}. The flow distribution can be solved by setting up continuity equations at each branch point in the array. From there, we apply periodic boundary conditions lateral to the flow direction and a constant pressure drop, ΔP, parallel to the flow direction across each array period. This system of equations thus reduces to solving the pressure at four nodes in the minimum unit cell, which are given by:

$$[ \begin{matrix}( 2 {R}_{B}^{- 1} + {R}_{A}^{- 1} + {R}_{T}^{- 1} ) & - 2 {R}_{B}^{- 1} & - ( {R}_{A}^{- 1} + {R}_{T}^{- 1} ) & 0 \\ - 2 {R}_{B}^{- 1} & ( 2 {R}_{B}^{- 1} + {R}_{A}^{- 1} + {R}_{T}^{- 1} ) & 0 & - ( {R}_{A}^{- 1} + {R}_{T}^{- 1} ) \\ - ( {R}_{A}^{- 1} + {R}_{T}^{- 1} ) & 0 & ( 2 {R}_{B}^{- 1} + {R}_{A}^{- 1} + {R}_{T}^{- 1} ) & - 2 {R}_{B}^{- 1} \\ 0 & - ( {R}_{A}^{- 1} + {R}_{T}^{- 1} ) & - 2 {R}_{B}^{- 1} & ( 2 {R}_{B}^{- 1} + {R}_{A}^{- 1} + {R}_{T}^{- 1} )\end{matrix} ] [ \begin{matrix}{P}_{i , j} \\ {P}_{i + 1 , j} \\ {P}_{i , j + 1} \\ {P}_{i + 1 , j + 1}\end{matrix} ] = Δ P [ \begin{matrix}{R}_{A}^{- 1} \\ {R}_{T}^{- 1} \\ - {R}_{A}^{- 1} \\ - {R}_{T}^{- 1}\end{matrix} ] \qquad ( 3 )$$

where P_{i,j}, P_{i,j+1}, and P_{i+1,j+1} are the four unique nodes in the unit cell. An infinite ladder network can be similarly modeled by replacing all instances of “2” with “1”, and by assigning j to 0 and j+1 to 1.

The pressures at each node can be solved by inverting Eq. (3) to yield a generic solution in terms of the pressure at an arbitrary point, in this case chosen as P_{i,j}:

$${P}_{i , j} = {P}_{i , j} \\ {P}_{i + 1 , j} = {P}_{i , j} - \frac{1}{2} \frac{{R}_{A}^{- 1} - {R}_{T}^{- 1}}{{R}_{A}^{- 1} + 2 {R}_{B}^{- 1} + {R}_{T}^{- 1}} Δ P \\ {P}_{i , j + 1} = {P}_{i , j} - \frac{1}{2} \frac{{R}_{B}^{- 1} + {R}_{A}^{- 1}}{{R}_{A}^{- 1} + 2 {R}_{B}^{- 1} + {R}_{T}^{- 1}} Δ P \\ {P}_{i + 1 , j + 1} = {P}_{i , j} - \frac{1}{2} Δ P \qquad ( 4 )$$

We can then determine the ratio of flow along the two lateral paths, Q_{B}, relative to the flow through the trap, Q_{T}, which are given by:

$$\frac{{Q}_{T}}{2 {Q}_{B}} = \frac{1}{2} \frac{{R}_{B} + {R}_{A}}{{R}_{T} - {R}_{A}} \qquad ( 5 )$$

Because the solution changes sign as a function of the relative magnitude of R_{A} and R_{T}, this result indicates that there are two regimes of fluid flow. When R_{T}>R_{A}, which is the typical scenario for previously studied trapping designs, the flow ratio is positive and approaches a singularity when R_{T} is nearly equal to R_{A}. This singularity defines a critical point where there is zero flow through the lateral branches, R_{B}, and all of the flow moves solely through the R_{A} and R_{T} paths, practically in straight lines. An alternative way to think of this phenomena is that the pressure at the adjacent nodes P_{i,j} and P_{i+1,j} are equal when R_{A} and R_{T} have equal resistance, leading to zero flow in the lateral branches.

The other flow regime, which has not previously been reported, occurs when R_{T}<R_{A}, which leads to the ratio in Eq. (5) becoming negative. This sign inversion is indicative that the flow through the lateral braches, Q_{B}, is actually assigned in the wrong direction. Thus, in this flow regime all fluid branches join to flow through the trap, which in theory should lead to nearly perfect trapping efficiency.

### Example 2—Simulation of the Filling Process as Cells are Introduced to a Mesh Fluid Network Comprising a Plurality of Trapping Features

A distribution of cells captured in a microfluidic mesh network can be modeled as a conditional probability tree describing the capture of n cells in m traps with the additional constraints that:

1) each trap can accept only one cell

2) each cell is captured permanently in that trap

3) the cells move in the direction from inlet to outlet (i.e., increasing row numbers)

We assume for each trial throw there is a success rate, q=[0,1], which describes the probability that an unoccupied trap captures a cell, while there is zero probability that an occupied trap captures a second cell (at least for the purposes of this first order model). Each row in the trap array is assumed to have M traps arranged in N rows with the trap array having dimensions of N×M. Finally, we assume that the probability of a cell captured in the i^{th} row is related to the percentage of occupied traps in that row, Cᵢ=[0,1] where Cᵢ=1 signifies a row that is completely saturated and Cᵢ=0 is a row that is empty. For ease of notation, we will also use the notation: C̅=1−C.

Now assume that Pᵢ is the probability that a cell is captured by the i^{th} row, whereas the probability that the cell is captured in the (i+1)^{th} row is reduced by the probability that the cell is not first captured by the previous i rows, i.e., 1−ΣPᵢ. The rates of capture by each row can then be modeled as:

$${P}_{1} = q {\overline{C}}_{1} \\ {P}_{2} = q {\overline{C}}_{2} ( 1 - {P}_{1} ) = q {\overline{C}}_{2} ( 1 - q {\overline{C}}_{1} ) \\ {P}_{3} = q {\overline{C}}_{3} ( 1 - {P}_{1} - {P}_{2} ) = q {\overline{C}}_{3} ( 1 - q {\overline{C}}_{1} - q {\overline{C}}_{2} ( 1 - q {\overline{C}}_{1} ) ) = q {\overline{C}}_{3} ( 1 - q {\overline{C}}_{2} ) ( 1 - q {\overline{C}}_{1} ) \\ … \\ {P}_{N} = q {\overline{C}}_{N} ( 1 - {\sum }_{i = 1}^{N - 1} {P}_{j} ) = q {\overline{C}}_{N} {\prod }_{i = 1}^{N - 1} ( 1 - q {\overline{C}}_{i} ) \qquad ( 6 )$$

The filling process in the array can be solved through an approximate rate equation. For example, assume during a discrete time interval that n cells are thrown into the array at a constant rate, γ, such that during a short time interval, Δt, the number of cells added to the array is n=γ Δt. This process leads to a first order rate equation for the first row given by:

$$Δ {\overline{C}}_{1} = - \frac{n}{M} {P}_{1} = - \frac{n}{M} q {\overline{C}}_{1} = \frac{γ Δ t}{M} q {\overline{C}}_{1} \\ \frac{d {\overline{C}}_{1}}{d t} = - \frac{γ}{M} q {\overline{C}}_{1} \qquad ( 7 )$$

where M is the number of traps in each row. The change in concentration of the other rows, Cᵢ for i>1 are similarly given by:

$$\frac{d {\overline{C}}_{N}}{d t} = - \frac{γ}{M} q {\overline{C}}_{N} {\prod }_{i = 1}^{N - 1} ( 1 - q {\overline{C}}_{i} ) \qquad ( 8 )$$

which is based on the capture probabilities derived above. These rate equations can be numerically solved with finite difference techniques and integrated as follows:

$$\frac{d {\overline{C}}_{j}}{d t} \approx \frac{{\overline{C}}_{j}^{t + 1} - {\overline{C}}_{j}^{t}}{Δ t} = - \frac{γ}{M} q {\prod }_{i = 1}^{j - 1} ( 1 - q {\overline{C}}_{i}^{t} ) or \\ {\overline{C}}_{j}^{t + 1} = {\overline{C}}_{j}^{t} - \frac{γ Δ t}{M} q {\overline{C}}_{j}^{t} {\prod }_{i = 1}^{j - 1} ( 1 - q {\overline{C}}_{i}^{t} ) \qquad ( 9 )$$

This system of equations is solved iteratively, first finding a solution at the t=1 time step and then updating the concentrations in all rows of the array. The concentrations at each position at time step, t=2, are then determined iteratively. The time step is kept small enough to avoid numerical artifacts.

### Example 3—Quantifying Cell Trapping Efficiency Vs. Resistance Ratio

These predictions were tested by designing and fabricating a variety of microfluidic trap architectures having different resistance ratios in the range of 0.25>R_{A}/R_{T}>1.5.

Microfluidic device fabrication: Microfluidic chips were fabricated on 6″ wafers using deep reactive ion etching (DRIE) to form the channel walls. Photoresist (Shipley 1813) was spun onto the wafers at 500 rpm for 5 s and 4000 rpm for 60 s, baked at 115° C. for 60 s, exposed to 80-100 mJ/cm² in a Karl Suss MA6 mask aligner, and then developed in Microposit MF319 developer for 30 s. The wafers were then thoroughly cleaned and etched to a depth of 15-20 μm in the DRIE (SPTS Pegasus Deep Silicon Etcher). The photoresist mask was then stripped and cleaned in piranha solution (3:1 H₂SO₄ to H₂O₂ at 200° C.). Next, a 10 μm thick layer of AZ 9260 photoresist was spun onto the backside of the wafer at 500 rpm for 5 s and 1800 rpm for 60 s, baked at 110° C. for 60 s, exposed to 4000 mJ/cm2 and developed for 300 s in AZ400K 1:4 developer. This layer was used to create through silicon vias to establish the inlets and outlets and dice the chips. The photoresist was then stripped and thoroughly cleaned as described previously. Finally, we anodically bonded borosilicate glass to the silicon microchannels at 300° C. for 3 hours. In total, each wafer yielded 36 devices (chips), which have dimensions of 30 mm×25 mm.

Microfluidic setup: Custom-made chip holders were machined in aluminum (Protolabs, MN), and comprised a bottom holder and a top viewing window. The bottom piece contained ¼″-28 threaded holes to allow for connection to be made to the chips with screw-in luer locks (Idex, Lake Forest, Ill.). The chip holders were also anodized (Surtronics, Raleigh, N.C.) so that they can be placed in the cell culture incubator for long durations. The chip holders were fixed onto a 3D printed stage adapter that was mounted inside a motor-controlled X-Y stage (ASI Instruments, Eugene, Oreg.) that was fastened to a Leica DMI 6000-B inverted fluorescent microscope that contains an automated focus drive, objective changer, and filter changer. Fluid was introduced to the chip with an Elvesys MK3 pressure controller (Paris, France) mounted at the outlet and driven by vacuum control.

High-throughput microscopy: We have developed custom Micro-Manager (open source microscopy software) codes to rapidly take images of each chamber within a microfluidic device. The algorithm involved first identifying 3 corners in the array to establish the equation of a plane, next creating a stage position list containing the XY position and optimal focal plane for each chamber, then taking images of each chamber with a Retiga 2000-R camera, and finally saving and naming the images in custom formats to render them compatible with the machine learning-based image processing algorithms.

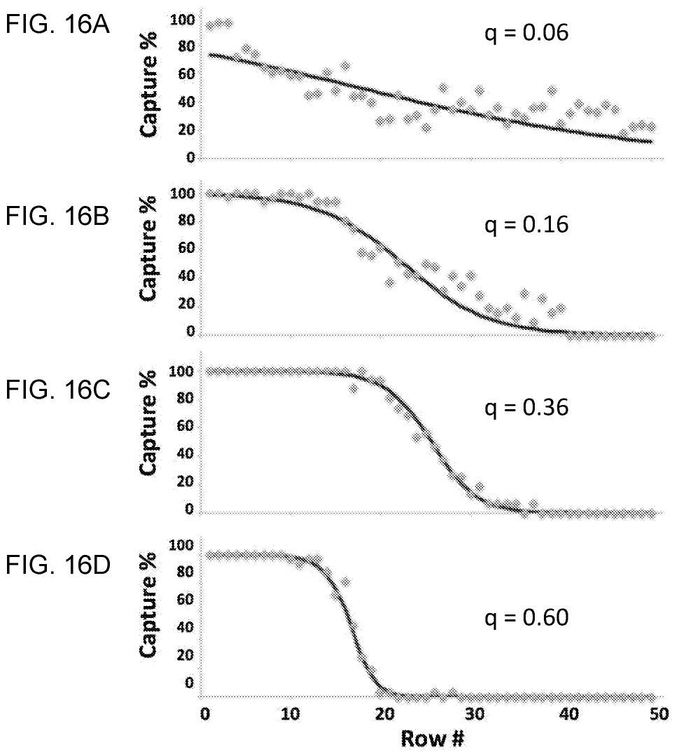

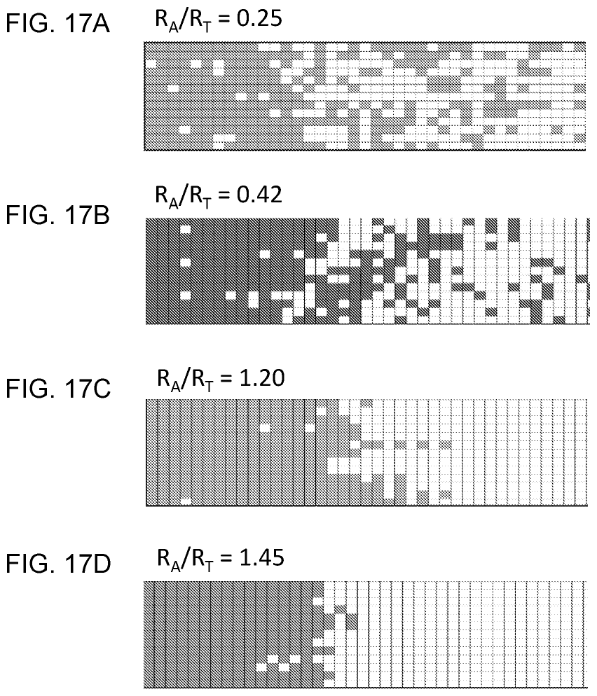

Results: In our experiments, we introduced a small enough number of cells such that the array would remain partially filled, enabling analysis of the filling fraction vs. the row number. The occupancy of each trap was identified with custom computer vision software. The experimental data was then fit to Eq. (9) with two fitting parameters, i.e., the trapping efficiency, q, and the total number of cells introduced. The results from one trial for each microfluidic device design are plotted in FIGS. 16A-16D as capture efficiency vs. row number for four different chips of increasing resistance ratio, along with an overlay of the best fit to Eq. (9). The best fit value of q is noted in each figure. FIGS. 17A-17D provide plots of the corresponding heat maps for the trapping distribution for each design. For low resistance ratios of R_{A}/R_{T}=0.25 or 0.42, the trapping efficiencies were determined to be 6% and 16% respectively. On the other hand, when the resistance ratio exceeded unity, the trapping efficiency increased dramatically and was as high as 70% in these experiments.

### Example 4—Long Term Cell Culture in Microfluidic Trap Arrays

Next, we demonstrated the ability to grow individual colonies at large enough scales to identify rare cell phenotypes. Cells were introduced to the array and transferred to the interior chambers with a gentle trap and transfer technique that is similar to previously reported methods (Dura, et al. (2014), “Deformability-based microfluidic cell pairing and fusion”, Lab. Chip 14:2783). Specifically, we first captured cells at the entrance of the traps, and next transferred the cells into the chambers with a brief pressure burst, causing the cells to squeeze through the narrow constrictions and move into the interior chambers. The widths of the constrictions were adjusted in the range of 3-6 microns, depending on the cell type the device was designed to capture. As a general rule, we found that a 1:3 ratio for the width of the trapping region compared to the diameter of the cell was ideal, and allowed cells to be reliable trapped and not squeezed through at low pressures (˜20 mbar), but rapidly transferred into the interior chambers at higher pressure (˜300 mbar). Flow control was achieved by adjusting negative pressure at the outlet, which allowed cells to be pipetted directly in the inlet reservoir with minimal losses from dead volume. Additionally, this approach allowed us to wash the cells from the inlet reservoir once the array was fully populated. The time required to populate the array depends on the cell concentration and the number of rows in the chip; however most designs had ˜50 rows and using cell concentrations on the order of 10⁶ cells/mL allowed us to fully populate the array within 5 minutes.

Once the cells were arrayed, we used our automated imaging algorithms written in Micro-Manager to take a high-resolution bright-field image of each chamber in the array. Thereafter, the chip was disconnected from the microscope and flow control apparatus, and transferred to a standard cell culture incubator where the cells were maintained for 7 days and beyond. While inside the incubator, flow was continually perfused in the chip either by gravity driven flow or with pressure controllers housed inside the incubator. In the case of gravity flow, we connected the inlet to a 5 mL syringe filled with media, while the outlet was connected to an unfilled syringe, and we consistently achieved flow rates of 0.25-0.50 mL per day through the chip, which was induced ˜5 mbar produced by the pressure head. In other experiments, we used pressure controllers to refresh the cell media, which allowed us to periodically rinse the chip every 10 minutes.

Cells were imaged twice per day and immediately returned to the incubator after each imaging cycle, requiring around 5-10 minutes for each chip. After 7 days, the imaging dataset was analyzed by custom computer vision software written in Python using a pre-trained Mask_RCNN image segmentation model. We developed a Tensorflow (machine learning)-based image segmentation algorithm to automatically align and crop the individual chambers from larger images, then determine the specific row and column addresses of each chamber, pick out each cell in the chamber at each time point, and then plot the number of cells in each colony.

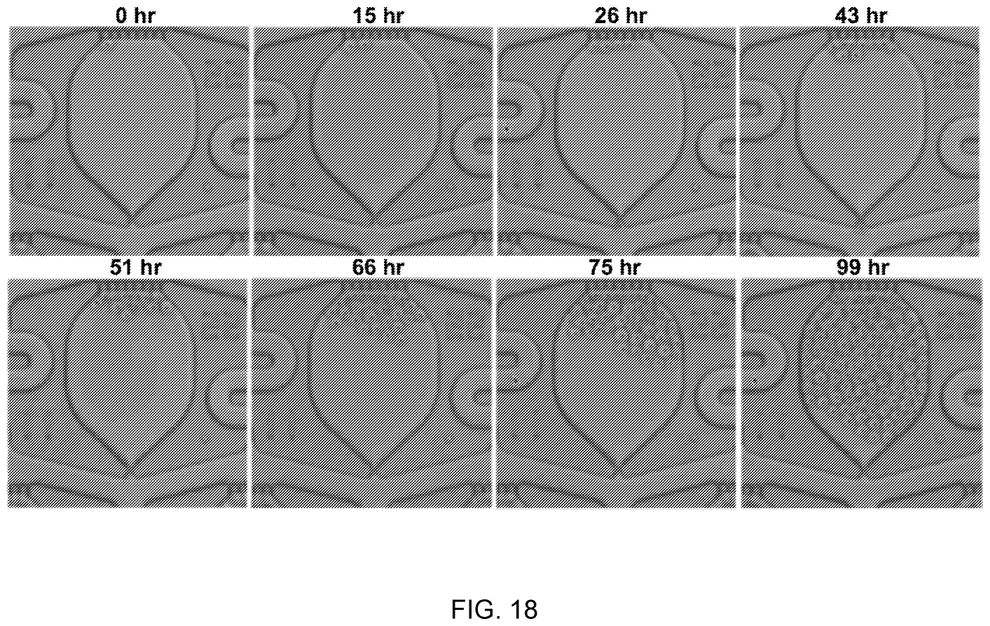

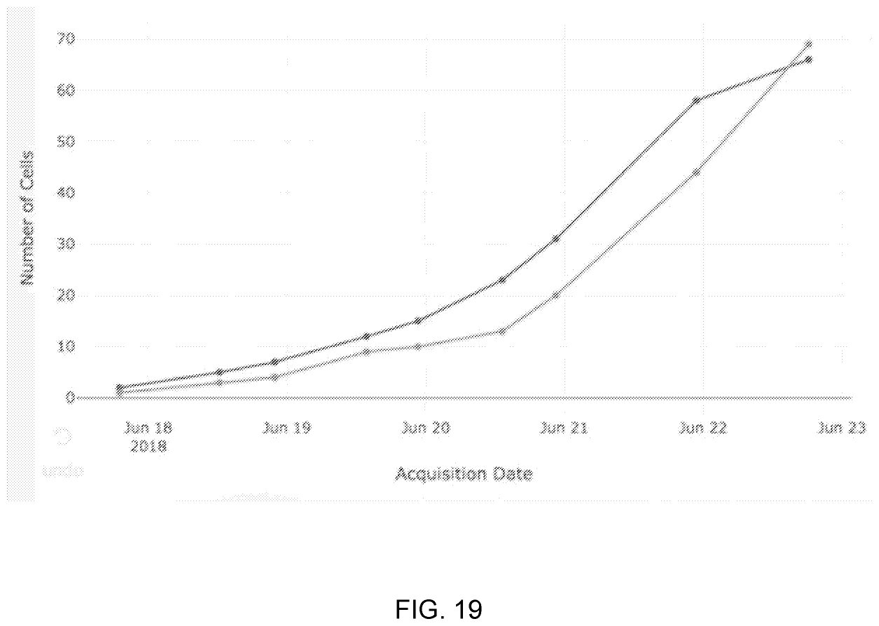

Time lapse images of proliferation from a K562 single cell clone in a control vehicle over 4 days are shown in FIG. 18, with red dots showing the cells identified with the computer vision algorithm. Mean doubling time was 13.5 hours, which is faster than the average doubling time of ˜20 hours for the population as a whole. The growth rate data for a couple of the faster growing clones are shown in FIG. 19.

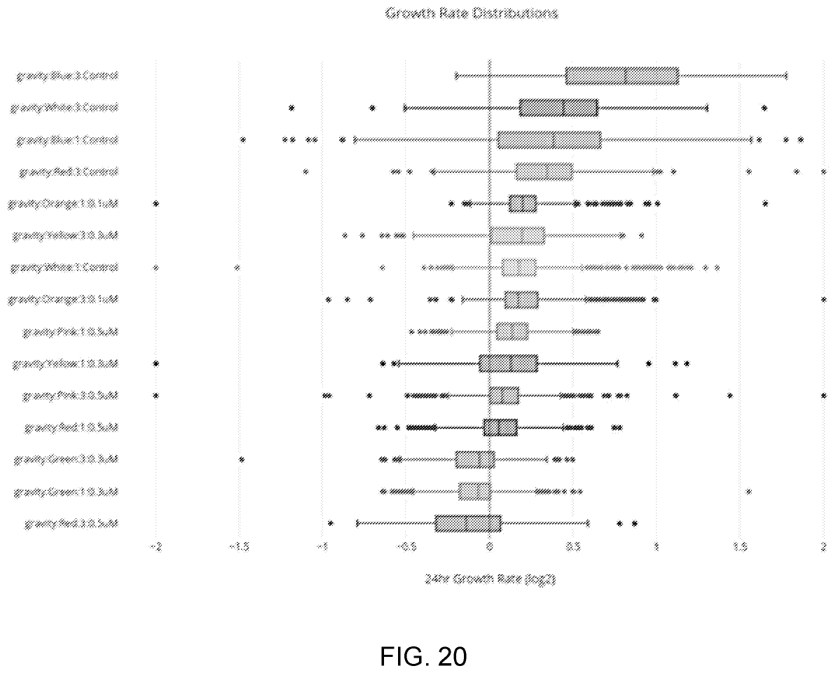

With this approach, we were able to automatically output the distribution of growth rates of each colony, and plot these in the form of heat maps or growth histograms. As one demonstration, we conducted an experiment in which K562 cells were grown in the presence of varying concentrations of Imatinib [0.1 μM, 0.3 μM, 0.5 μM] or control. The growth rate distributions are plotted in FIG. 20, showing the expected trends of decreased growth rate with increasing drug concentration. For each distribution, the outlier drug resistant cells could be clearly picked out from these datasets.

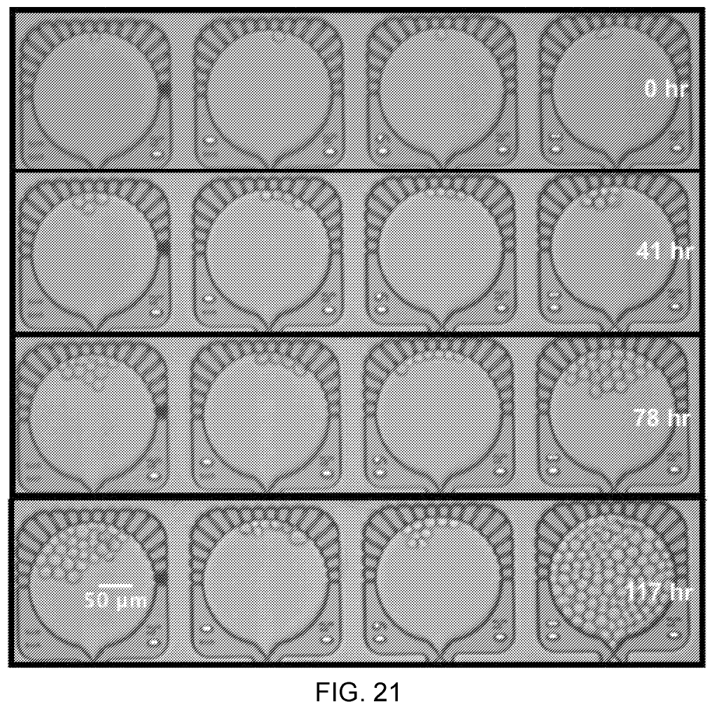

FIG. 21 shows a series of time lapse images of four colonies growing inside adjacent chambers.

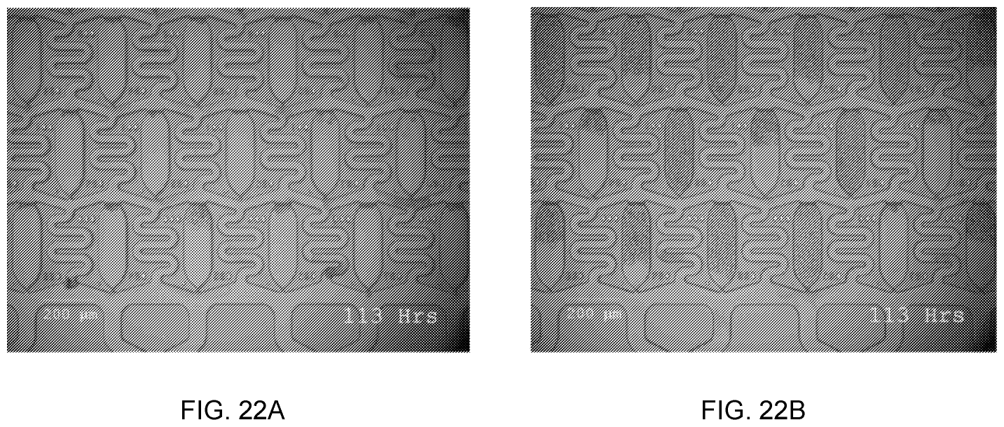

FIGS. 22A and 2B show images of MOLM 13 cells grown in the presence of Quizartinib (a small molecule inhibitor of receptor tyrosine kinases that is currently under development for the treatment of acute myeloid leukemia) (FIG. 22A) or a control medium (FIG. 22B). A single clone is observed to grow out in the presence of the drug.

### Example 5—Other Examples

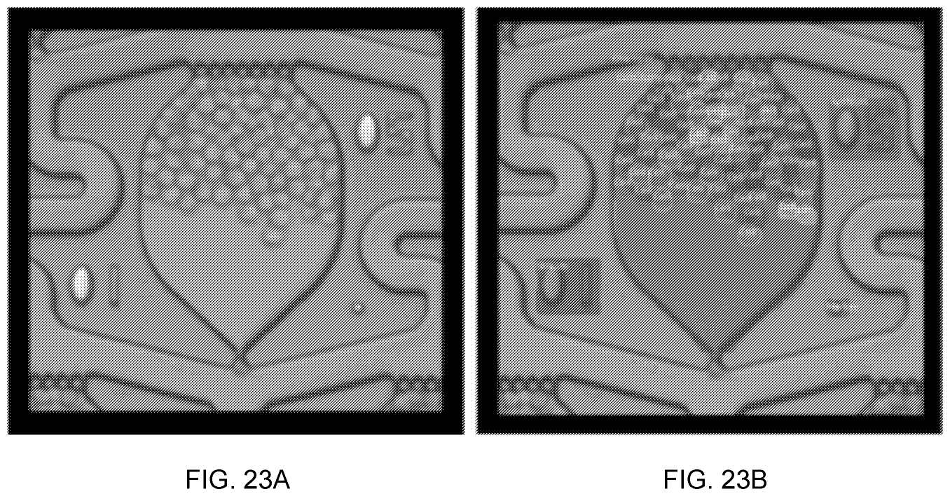

FIGS. 23A and 23B illustrate the use of image segmentation conducted using machine learning algorithms to identify individual cells as well as identifiers and markers on the microfluidic chip. FIG. 23A: bright-field image. FIG. 23B: a computer-generated color image is overlaid on the bright-field image and shows the identification of markers on the chip, and different instances of cells that have been classified using a machine learning-based analysis, the boundaries of the individual cells, and quality scores of the degree of confidence in the prediction of whether the object detected is a cell.

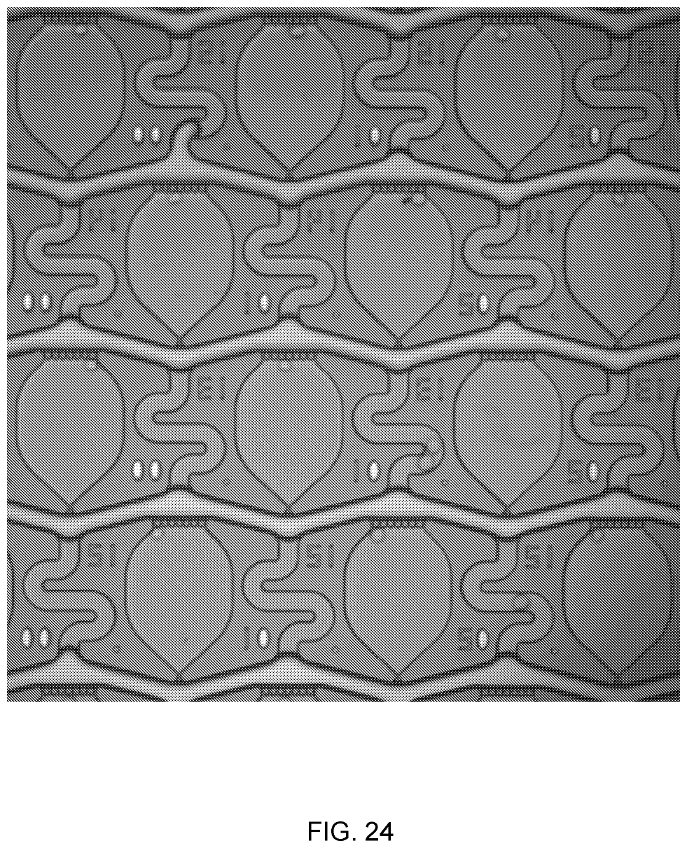

FIG. 24 shows and image of an array of single cells trapped within microfluidic chambers, after which air is blown through the fluid channels to seal the chambers.

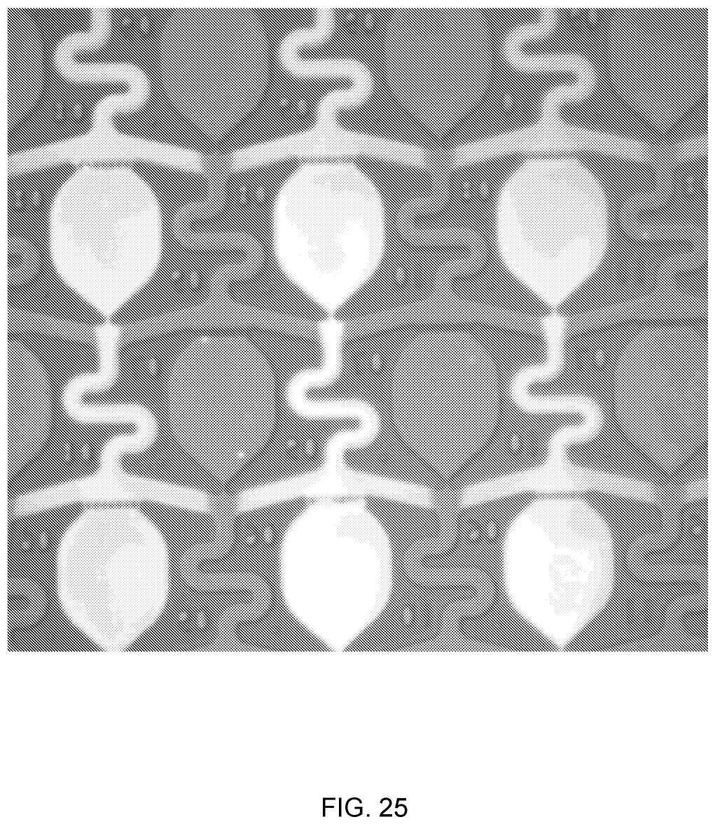

FIG. 25 shows an overlay of fluorescent and bright-field images that shows the hybridization of fluorescently-labeled target probes to oligonucleotide capture probes that are patterned inside the microfluidic chips.

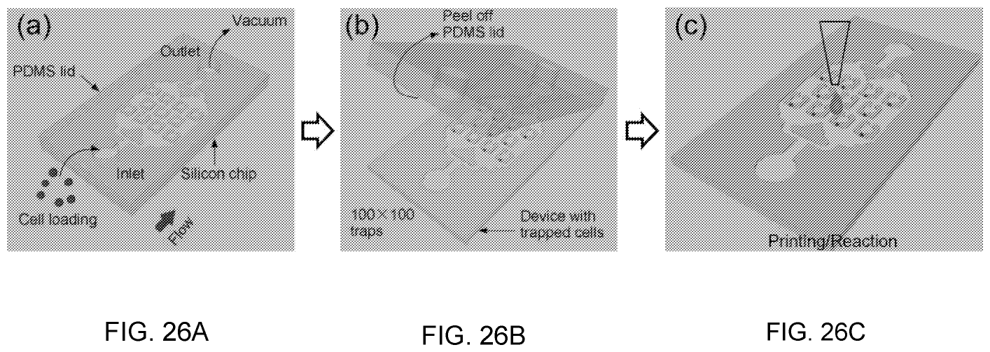

FIGS. 26A-26C illustrate a process for forming single cell arrays. Single cell arrays are formed by flowing cells into an array along with a curable hydrogel (FIG. 26A), after which the lid can be peeled away (FIG. 26B) to provide access to the sample (FIG. 26C).

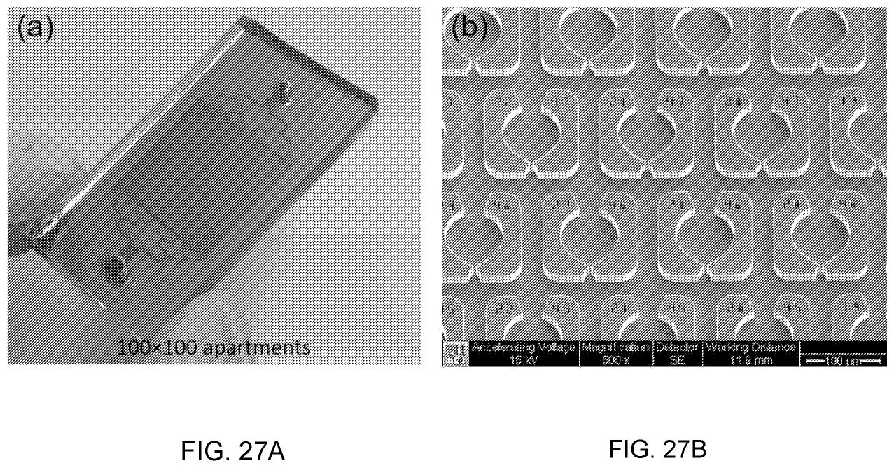

FIGS. 27A and 27B provide a non-limiting example of a microfluidic device comprising multiple trapping features for the capture of single cells or other objects suspended in a fluid. FIG. 27A: photograph of a microfluidic device comprising a 100×100 array of trapping features and microfluidic chambers. FIG. 27B: micrograph of the trapping features and fluid chambers within a microfluidic device of the present disclosure.

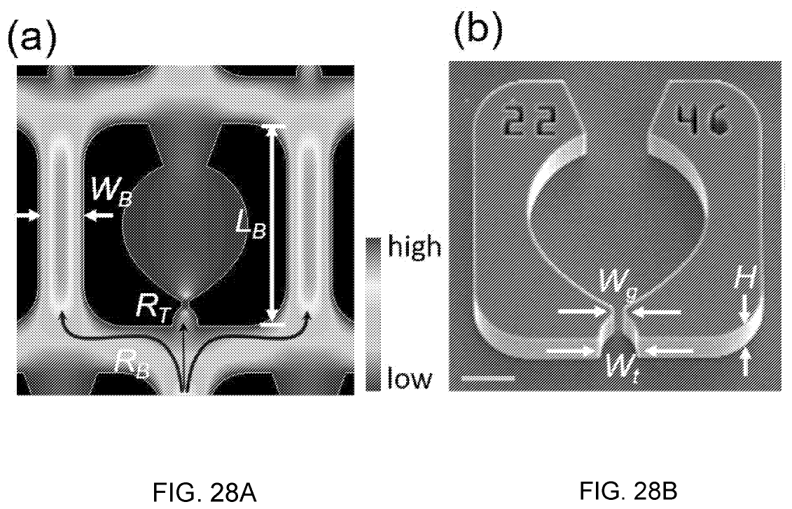

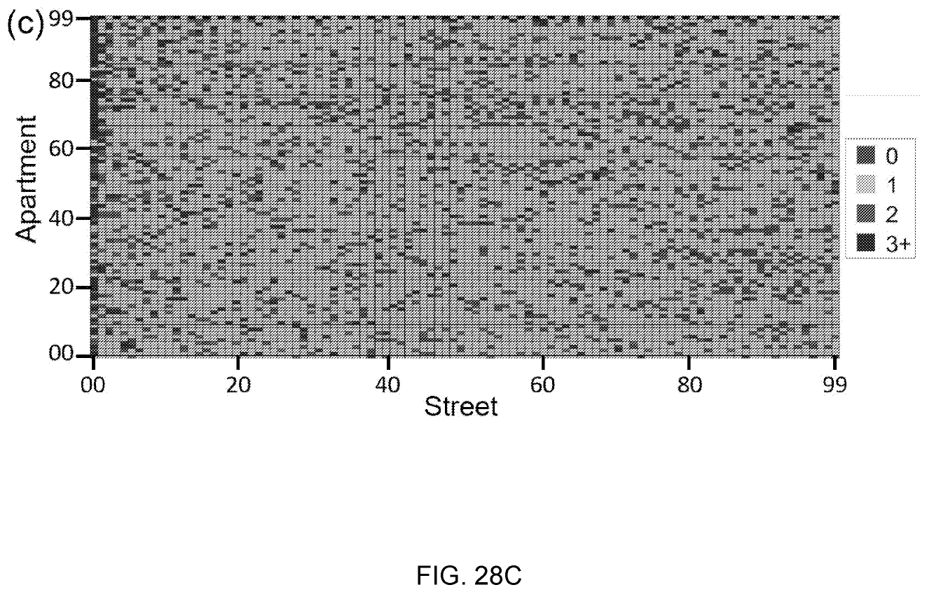

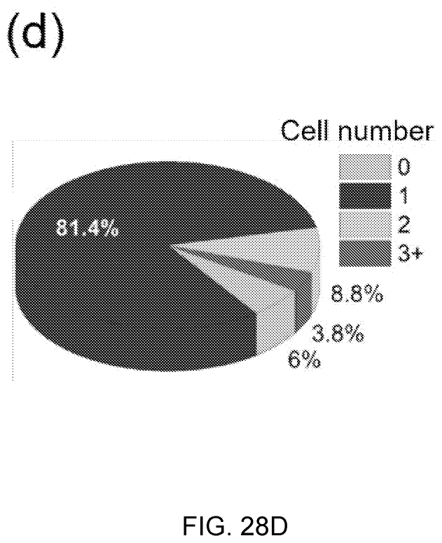

FIGS. 28A-28D provide examples of the flow profile through a trap for a low efficiency trapping device that was used in proof-of-principle work, as well as data for single cell trapping efficiency. FIG. 28A: calculated fluid flow velocity through a single trap of the device. FIG. 28B: micrograph showing a single trap of the device. FIG. 28C: heatmap showing the single cell trapping efficiency for the 10,000 compartments within the device. FIG. 28D: pie chart showing the distribution of microfluidic chambers within which 0, 1, 2, or 3 or more cells were trapped.

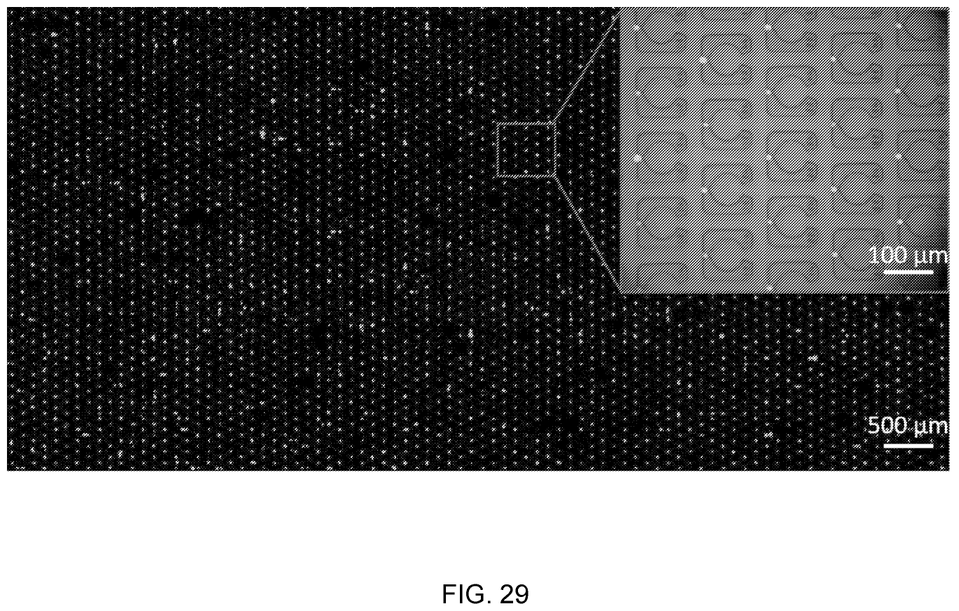

FIG. 29 shows a stitched fluorescent image of a cell array (cells are labeled with a FITC cell tracker dye). Inset: enlarged overlay of fluorescent and bright-field images showing individual cells trapped within the device.

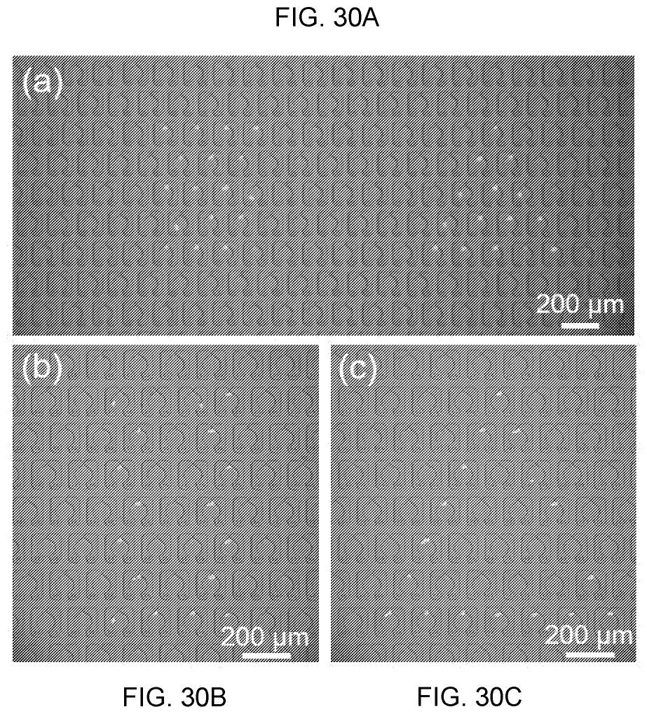

FIGS. 30A-30C show non-limiting examples of images that demonstrate the ability to print chemicals to specific cells in the array, which is made possible by the open architecture of the microfluidic device. FIG. 30A: two side by side patterns printed within a single cell array using a fluorescent label. FIG. 30B: pattern printed to specific cells within a cell array using a fluorescent label. FIG. 30C: pattern printed to specific cells within a cell array using a fluorescent label.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

What is claimed is:

1. A microfluidic device comprising:
a) a plurality of weir-traps disposed between, and in fluid communication with, at least one fluid inlet and at least one fluid outlet, wherein each weir-trap is configured to retain an object suspended in a fluid passing through the microfluidic device, and wherein:
i) each weir-trap comprises a constriction in at least one dimension that is less than about one third of a smallest dimension of the object; and
ii) a ratio of a fluidic resistance of a fluid flow path that bypasses a weir-trap to that for a fluid flow path passing through the weir-trap is at least 0.4.

2. The microfluidic device of claim 1, wherein the ratio of fluidic resistance is at least 0.75.

3. The microfluidic device of claim 1 or claim 2, wherein the ratio of fluidic resistance is at least 1.0.

4. The microfluidic device of any one of claims 1 to 3, wherein the ratio of fluidic resistance is at least 1.25.

5. A microfluidic device comprising:
a) a plurality of weir-traps disposed between, and in fluid communication with, at least one fluid inlet and at least one fluid outlet, wherein each weir-trap is configured to retain an object suspended in a fluid passing through the microfluidic device, and wherein:
i) each weir-trap comprises an entrance region, an interior region, and an exit region that collectively constitute an interior fluid flow path through the weir-trap that has a fluidic resistance, R_{T};
ii) each weir-trap in a majority of the weir-traps is in fluid communication with one long bypass fluid flow channel having a fluidic resistance, R_{A}, and with one or two short bypass fluid flow channels each having a fluidic resistance that is less than R_{A}, wherein each bypass fluid flow channel connects the exit region of the weir-trap to the entrance region of another weir-trap; and
iii) a ratio R_{A}/R_{T} is at least 1.0.

6. A microfluidic device comprising:
a) a plurality of weir-traps disposed between, and in fluid communication with, at least one fluid inlet and at least one fluid outlet, wherein each weir-trap is configured to retain an object suspended in a fluid passing through the microfluidic device, and wherein:
i) each weir-trap comprises an entrance region, an interior region, and an exit region that collectively constitute an interior fluid flow path through the weir-trap that has a fluidic resistance, R_{T};
ii) each weir-trap in a majority of the weir-traps is in fluid communication with one long bypass fluid flow channel having a fluidic resistance, R_{A}, and with one or two short bypass fluid flow channels each having a fluidic resistance that is less than R_{A}, wherein each bypass fluid flow channel connects the exit region of the weir-trap to the entrance region of another weir-trap; and
iii) fluid flows through an adjacent short bypass channel in a first direction if a weir-trap is unoccupied, and in a second direction if the weir-trap is occupied by an object.

7. The microfluidic device of claim 5 or claim 6, wherein the ratio R_{A}/R_{T} is at least 1.1.

8. The microfluidic device of any one of claims 5 to 7, wherein the ratio R_{A}/R_{T} is at least 1.2.

9. The microfluidic device of any one of claims 5 to 8, wherein the ratio R_{A}/R_{T} is at least 1.3.

10. The microfluidic device any one of claims 5 to 9, wherein the ratio R_{A}/R_{T} is at least 1.4.

11. The microfluidic device of any one of claims 5 to 10, wherein the ratio R_{A}/R_{T} is at least 1.45.

12. The microfluidic device of any one of claims 5 to 11, wherein each weir-trap comprises at least one constriction that has a spatial dimension that is less than about one half of the smallest dimension of the object.

13. The microfluidic device of any one of claims 5 to 12, wherein each weir-trap comprises at least one constriction that has a spatial dimension that is less than about one third of the smallest dimension of the suspended objects.

14. The microfluidic device of any one of claims 5 to 13, wherein each weir-trap comprises at least one constriction that has a spatial dimension that ranges from about 1.5 μm to about 6 μm.

15. The microfluidic device of any one of claims 5 to 14, wherein the ratio R_{A}/R_{T} is at least 1.2 and a capture probability for an individual weir-trap retaining a suspended object on first contact is at least 0.36.

16. The microfluidic device of any one of claims 5 to 15, wherein the ratio R_{A}/R_{T} is at least 1.45 and a capture probability for an individual weir-trap retaining a suspended object on first contact is at least 0.60.

17. The microfluidic device of any one of claims 5 to 16, wherein each weir-trap comprises a frit structure within the exit region, and wherein the frit structure comprises one or more constrictions that have a spatial dimension that is smaller than the smallest dimension of the suspended objects.

18. The microfluidic device of any one of claims 1 to 17, wherein the plurality of weir-traps comprises at least 100 weir traps.

19. The microfluidic device of any one of claims 1 to 18, wherein the plurality of weir-traps comprises at least 1,000 weir traps.

20. The microfluidic device of any one of claims 1 to 19, wherein the plurality of weir-traps comprises at least 10,000 weir traps.

21. The microfluidic device of any one of claims 1 to 20, wherein the plurality of weir-traps comprises at least 100,000 weir traps.

22. The microfluidic device of any one of claims 1 to 21, wherein a pre-saturation trapping efficiency for trapping the suspended objects is at least 20%.

23. The microfluidic device of any one of claims 1 to 21, wherein a pre-saturation trapping efficiency for trapping the suspended objects is at least 50%.

24. The microfluidic device of any one of claims 1 to 23, wherein a pre-saturation trapping efficiency for trapping the suspended objects is at least 80%.

25. The microfluidic device of any one of claims 1 to 24, wherein a pre-saturation trapping efficiency for trapping the suspended objects is at least 90%.

26. The microfluidic device of any one of claims 1 to 25, wherein a pre-saturation trapping efficiency for trapping the suspended objects is at least 95%.

27. The microfluidic device of any one of claims 1 to 26, further comprising: b) a removable lid.

28. The microfluidic device of any one of claims 1 to 27, wherein an interior region of one or more weir-traps comprises a unique molecular identifier that may be bound to or hybridized to molecular components of a cell upon lysis of a cell within the interior region of a weir-trap.

29. A method for trapping objects suspended in a fluid, the method comprising:
a) providing a microfluidic device of any one of claims 1 to 27; and
b) flowing a fluid comprising the objects through the microfluidic device to trap objects in one or more of the plurality of weir-traps.

30. The method of claim 29, wherein each weir-trap comprises a frit structure within an exit region, and wherein the frit structure comprises one or more constrictions that have a spatial dimension that is smaller than the smallest dimension of the objects.

31. The method of claim 29 or claim 30, wherein the flowing in (b) is performed at a first hydrodynamic pressure, thereby trapping an object in a constriction in an entrance region of one or more weir-traps.

32. The method of claim 31, wherein the objects comprise deformable objects, and wherein the method further comprises subjecting the object(s) trapped in the constriction in the entrance region(s) of one or more weir-traps to a second hydrodynamic pressure that is higher than the first hydrodynamic pressure, thereby forcing the deformable object(s) through the constriction in the entrance region(s) and into an interior region of the one or more weir-traps.

33. The method of claim 31 or claim 32, wherein the first hydrodynamic pressure ranges from about 1 to about 100 mbar.

34. The method of claim 32 or claim 33, wherein the second hydrodynamic pressure ranges from about 100 mbar to about 1,000 mbar.

35. The method of any one of claims 32 to 34, wherein the ratio of the second hydrodynamic pressure to the first hydrodynamic pressure ranges from about 10× to about 20×.

36. The method of any one of claims 29 to 35, wherein the objects are cells or beads.

37. The method of any one of claims 32 to 36, wherein the flowing in (b) is repeated at least once, thereby allowing at least two objects to be confined within the interior region(s) of one or more weir-traps.

38. The method of claim 37, wherein the flowing in (b) is repeated at least once using a fluid that comprises the same objects as that used in the first instance.

39. The method of claim 37, wherein the flowing in (b) is repeated at least once using a fluid that comprises different objects than that used in the first instance.

40. The method of any one of claims 37 to 39, wherein the at least two objects confined within the interior region(s) of one or more weir-traps comprise at least two of the same cells, at least two different cells, at least two of the same beads, at least two different beads, or at least one cell and one bead.

41. The method of any one of claims 29 to 40, further comprising sealing the plurality of weir-traps by flowing an immiscible fluid through the microfluidic device.

42. The method of claim 41, wherein the immiscible fluid is oil or air.

43. The method of any one of claims 32 to 42, wherein the objects are cells, and wherein the cells are cultured within the interior region(s) of the one or more weir-traps for a period of one or more days.

44. The method of claim 43, wherein the cells are cultured within the interior region(s) of the one or more weir-traps for a period of one or more weeks.

45. The method of claim 43, wherein the cells are cultured within the interior region(s) of the one or more weir-traps for a period of one or more months.

46. The method of any one of claims 32 to 45, wherein the objects are cells, and wherein the method further comprises the use of an imaging technique to phenotype cells within the interior region(s) of the one or more weir-traps.

47. The method of claim 46, wherein the imaging technique is selected from the group consisting of bright-field imaging, fluorescence imaging, two-photon fluorescence imaging, or any combination thereof.

48. The method of any one of claims 32 to 47, wherein the interior regions of the plurality of weir-traps each comprise unique molecular identifiers that may be bound or hybridized to molecular components of a cell upon lysis of a cell within the interior region of a weir-trap.

49. The method of claim 48, wherein the molecular components comprise proteins, peptides, DNA molecules, RNA molecules, mRNA molecules, or any combination thereof.

50. The method of claim 49, wherein the unique molecular identifiers are used to perform DNA sequencing, gene expression analysis, or chromatin analysis.

51. The method of claim 50, wherein an externally-applied electric field is used to facilitate hybridization of nucleic acid molecular components to the unique molecular identifiers.

52. The method of any one of claims 32 to 51, wherein the microfluidic device further comprises a removable lid.

53. The method of claim 52, wherein the deformable objects are cells, and wherein following the trapping of cell(s) in the interior region(s) of one or more weir-traps, a biocompatible hydrogel is infused into the microfluidic device and allowed to polymerize.

54. The method of claim 53, wherein following the polymerization of the hydrogel, the lid of the microfluidic device is removed to allow access to the trapped cells.

55. The method of any one of claims 53 to 54, wherein the biocompatible hydrogel is used to confine the genomic material of a trapped cell upon lysis of the cell.
